(19) United States
(12) Patent Application Publication
(10) Pub. No.: US 2021/0087252 A1
(43) Pub. Date: Mar. 25, 2021

(54) Mutant IDH1 Specific T Cell Receptor

(71) Applicants: Berkeley Lights, Inc., Emeryville, CA (US); The Regents of the University of California, Oakland, CA (US)

(72) Inventors: Hideho Okada, San Francisco, CA (US); Duane Smith, Berkeley, CA (US); Payal Watchmaker, San Francisco, CA (US); Yelena Bronevetsky, Alameda, CA (US); Ryosuke Naka, San Francisco, CA (US); Guido K. Stadler, San Francisco, CA (US); Xiaohua Wang, Pomona, NY (US); Kevin T. Chapman, Emeryville, CA (US)

(73) Assignees: Berkeley Lights, Inc., Emeryville, CA (US); The Regents of the University of California, Oakland, CA (US)

(21) Appl. No.: 16/987,835

(22) Filed: Aug. 7, 2020

Related U.S. Application Data
(63) Continuation of application No. PCT/US2019/017208, filed on Feb. 8, 2019.
(60) Provisional application No. 62/628,992, filed on Feb. 10, 2018.
(60) Provisional application No. 62/628,986, filed on Feb. 10, 2018.

(51) Int. Cl. C07K 14/725; C12N 5/0783; C12N 15/63

(52) U.S. Cl. CPC C07K 14/7051; C12N 5/0636; C12Y 101/01041; C07K 2317/565; C12N 15/63

(57) ABSTRACT

This disclosure relates to the production and use of an isolated, purified and/or recombinant T cell receptor (TCR) that specifically binds to a mutant IDH1 protein, or a fragment thereof, wherein the mutant IDH1 protein or fragment thereof comprises an R132H mutation.

This application is a continuation of International Patent Application No. PCT/US2019/017208, filed Feb. 8, 2019, which claims the benefit of priority of U.S. Provisional Application No. 62/628,992, filed Feb. 10, 2018, and U.S. Provisional Application No. 62/628,986, filed Feb. 10, 2018, each of which are incorporated by reference for all purposes.

This invention was made with government support under grant no. R21 NS093654 awarded by the National Institutes of Health. The government has certain rights in the invention.

The present application contains a Sequence Listing in electronic format. The Sequence Listing is provided as a file entitled “2019-02-07_01149-0003-00PCT_Seq_List_ST25.txt” created on Feb. 7, 2019, which is 29 KB in size, and is incorporated by reference herein.

### BACKGROUND

Gliomas are the most common type of primary brain tumors. This group of tumors includes a number of specific histologies, the most common of which are astrocytomas, oligodendrogliomas, and ependymomas.

Isocitrate dehydrogenase 1 (IDH1) catalyzes the oxidative decarboxylation of isocitrate to alpha-ketoglutarate by coupling the reaction to the reduction of NADP+ to NADPH. IDH1 mutations occur at a high frequency in WHO grade II and III diffuse gliomas, and 93% of all IDH1 mutations are characterized by the amino acid change R132H (Hartmann 2009, Acta Neuropathol. 118(4):469-74). The arginine at position 132 of the amino acid sequence of IDH1 is located in an evolutionarily conserved isocitrate-binding site.

Although there is evidence for cytotoxic T cell responses against mutant IDH1 in patients, tolerance to self-antigens can be a limiting factor in generating highly avid T cells capable of killing tumor cells. An alternate approach involves adoptive cell therapy, which uses T cells that have been engineered to potently recognize and attack tumors by expressing T cell receptors (TCRs) that specifically bind to mutant IDH1. There is a continuing need, however, to identify TCRs that can be used to produce engineered T cells suitable for treatment of gliomas and other brain tumors.

### SUMMARY OF THE INVENTION

Isolated, purified and/or recombinant T cell receptors (TCR), or fragments thereof, that specifically bind to an IDH1 peptide fragment containing a mutation are provided. In some embodiments, the mutant IDH1 peptide fragment comprises an R132H mutation. In some embodiments, the TCR, or fragment thereof, comprises one or more (e.g., one, two, three, four, five, or six) amino acid sequences set forth in SEQ ID NOs: 4, 6, 8, 12, 14, 16, 20, 22, 24, 28, 30, 32, 36, 38, and 40, and variants thereof. In some embodiments, the TCR, or fragment thereof, comprises an amino acid sequence set forth in SEQ ID NO: 2, 18, or 34, or a variant thereof; and/or an amino acid sequence set forth in SEQ ID NO: 10 or 26, or a variant thereof.

Also provided are: nucleic acid molecules encoding any of the TCRs disclosed herein, or a fragment thereof; recombinant expression vectors that contain a nucleic acid molecule encoding any of the TCRs disclosed herein, or a fragment thereof; host cells that contains any of the nucleic acid molecules or recombinant expression vectors disclosed herein and/or expresses any of the TCRs disclosed herein, or a fragment thereof; and methods of generating such host cells. The host cells can, for example, be T cells.

Also provided are: pharmaceutical compositions comprising a host cell disclosed herein and/or a TCR disclosed herein, or a fragment thereof; and methods of treating a patient by administering to the patient such a pharmaceutical composition. The patient, for example, can be suffering from a cancer, such as a glioma (e.g., a diffuse glioma, which may be a diffuse low-grade glioma).

These and other features and advantages of the disclosed compositions and methods will be set forth or will become more fully apparent in the description that follows, including the examples, and in the appended claims. Furthermore, the features and advantages of the described compositions and methods may be learned by the practice of the inventions.

Section headings are provided solely for the convenience of the reader and do not limit the scope of the disclosure in any way. In the event that any material incorporated by reference conflicts with the express content of this disclosure, the express content controls.

### BRIEF DESCRIPTION OF THE DRAWINGS

FIG. 1 depicts pie charts showing the relative abundance of different types of TRAV and TRBV alleles detected upon sequencing T cell populations selectively expanded in the presence of IDH1 R132H mutant peptide according to certain embodiments. A1, A3, A5, C1, C3, and C5 designate individual wells from which the expanded T cell populations were obtained.

FIG. 2 depicts FACS results of Jurkat 76 cells transfected with the 26-9 TCR clone, according to one embodiment.

FIGS. 3A-3B depict bar graphs showing significant elevation of TNF-α (3A) and IFN-γ (3B) levels in cell culture supernatants from transfected CD4⁺ T cells expressing the 26-9 TCR clone, when exposed to IDH1 R132H mutant peptide as compared to control IDH1 wild-type peptide, according to one embodiment. FIG. 3C depicts a bar graph showing significant elevation of IL-2 levels in cell culture supernatant from transfected CD4+ T cells expressing the 26-9 TCR clone, when exposed to IDH1 R132H mutant peptide as compared to control IDH1 wild-type peptide.

FIG. 4 depicts a bar graph showing significant elevation of TNF-α levels in cell culture supernatants from transfected CD4⁺ T cells expressing the 16-9 TCR clone, when exposed to IDH1 R132H mutant peptide (“mut pep”) as compared to control IDH1 wild-type peptide (“wt pep”), according to one embodiment.

### DETAILED DESCRIPTION

### Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X.”

The terms “nucleic acid molecule”, “nucleic acid” and “polynucleotide” may be used interchangeably and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues and are not limited to a minimum length. Such polymers of amino acid residues may contain natural or non-natural amino acid residues and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present disclosure, a “polypeptide” refers to a protein which includes modifications, such as deletions, additions and substitutions (generally conservative in nature), to the native sequence, as long as the protein maintains the desired activity. These modifications may be deliberate, as through site-directed mutagenesis, or may be accidental, such as mutations introduced by host organisms used to clone DNA or express proteins or errors due to PCR amplification.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily and/or with greater duration than it binds to other substances. For example, an antibody that specifically or preferentially binds to a PD-1 epitope is an antibody that binds this epitope with greater affinity, avidity, more readily and/or with greater duration than it binds to other PD-1 epitopes or non-PD-1 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99% pure.

The term “Isocitrate dehydrogenase Type 1” or “IDH1” as used herein refers to an enzyme which is physiologically involved in the citric acid cycle in that it catalyzes the oxidative decarboxylation of isocitrate whereby alpha-ketoglutarate and CO₂ are produced. The reaction requires the conversion of NAD⁺ to NADH. Another isoform of the enzyme disclosed herein catalyzes the same reaction in the cytosol as well as in mitochondria and peroxisomes using NADP⁺ as a cofactor rather than NAD⁺. IDH1 as referred to herein, preferably, is human IDH1 having an amino acid sequence as disclosed in Kim et al. (Kim 1995, Biochem J 308(Ptl): 63-68) or as available under NCBI/Genbank accession numbers CAG46496.1, GI: 49456351; CAG38738.1, GL49168486; CAG38553.1, GI: 49065470; or AAH12846.1, GI: 15277488. The term, however, also encompasses variants of said human IDH1 characterized by the aforementioned specific amino acid sequence. Such variants may be orthologs, paralogs or homologs, in general.

“Fragment” means an immunogenically effective subset of the amino acid sequence that comprises a T cell receptor (TCR). The term is intended to include such fragments in conjunction with or combined with additional sequences or moieties, for example, where the peptide is coupled to other amino acid sequences or to a carrier.

The term “subject” as used herein relates to animals, preferably mammals, and, more preferably, humans. In some embodiments, the methods disclosed herein will be applied to subjects suspected to either suffer from any of the aforementioned cancer types in light of clinically apparent symptoms or subjects suspected to suffer from said cancer due to a potential increased predisposition.

### IDH1 Specific T Cell Receptor

### Functionality

Disclosed herein, in some embodiments, is an isolated, purified and/or recombinant T cell receptor (TCR) that specifically binds to a mutant IDH1 protein, or a fragment thereof. In some embodiments, the mutant IDH1 protein or fragment thereof comprises an R132H mutation. In some embodiments, the TCR disclosed herein does not bind to an IDH1 protein comprising an arginine at amino acid position 132 (i.e., an arginine instead of a histidine at position 132 of SEQ ID NO: 42) or fragment thereof comprising the arginine at position 132 (i.e., position 132 according to the full-length IDH1 sequence). In some embodiments, the disclosed TCR polypeptide or fragment thereof binds to an R132H mutant of IDH1 or a fragment of IDH1 comprising the R132H mutation when the R132H mutant of IDH1 or a fragment of IDH1 comprising the R132H mutation is bound to a major histocompatibility (MHC) molecule.

In some embodiments, the TCRs disclosed herein comprise one or more immature TCR chains comprising a leader sequence, such as a signal sequence, or one or more mature chains in which the leader sequence has been cleaved off. As one of ordinary skill in the art appreciates, the signal sequence of a TCR chain comprises the amino acids at the N-terminus which together serve as a signal to transport the TCR to the plasma membrane and which amino acids are cleaved off to yield the mature form of the TCR.

In some embodiments, disclosed herein is a TCR complex comprising at least two TCRs as disclosed herein.

It is contemplated that any of the TCRs, proteins, or polypeptides disclosed herein can be isolated, substantially pure, and/or recombinantly expressed.

### Exemplary Polypeptide Sequences

Disclosed herein, in some embodiments, is a protein comprising a TCR alpha chain, or a fragment thereof, and/or a TCR beta chain or a fragment thereof, wherein said alpha chain or beta chain or fragment thereof comprises one or more amino acid sequences represented by SEQ ID NOs: 4, 6, 8, 12, 14, 16, 20, 22, 24, 28, 30, 32, 36, 38, and 40, and variants thereof. In some embodiments, the protein comprises one or more amino acid sequences represented by SEQ ID NOs: 2, 10, 18, 26, and 34, and variants thereof. Another embodiment disclosed herein provides protein comprising one or more amino acid sequences represented by SEQ ID NOs: 8, 16, 24, 32, and 40, and variants thereof.

The TCR proteins disclosed herein can comprise an α and a β chain. For example, a TCR protein disclosed herein can comprise a first polypeptide chain comprising the amino acid sequence of SEQ ID NO: 2, 18, or 34 (an α chain), and a second polypeptide chain comprising the amino acid sequence of SEQ ID NO: 10 or 26 (a β chain). A TCR protein disclosed herein can, for example, comprise a first polypeptide chain comprising the amino acid sequence of SEQ ID NO: 2 (an α chain) and a second polypeptide chain comprising the amino acid sequence of SEQ ID NO: 10 (a β chain). A TCR protein disclosed herein can, for example, comprise a first polypeptide chain comprising the amino acid sequence of SEQ ID NO: 34 (an α chain) and a second polypeptide chain comprising the amino acid sequence of SEQ ID NO: 10 (a β chain). A TCR protein disclosed herein can, for example, comprise a first polypeptide chain comprising the amino acid sequence of SEQ ID NO: 18 (an α chain) and a second polypeptide chain comprising the amino acid sequence of SEQ ID NO: 26 (a β chain). In some embodiments, the TCR disclosed herein comprises SEQ ID NO: 2, 10, 18, 26, or 34. In some embodiments, a TCR disclosed herein comprises any alpha chain disclosed herein with any other beta chain disclosed herein.

In some embodiments, a TCR or protein disclosed herein comprises at least one sequence selected from SEQ ID NOs: 4, 6, 8, 12, 14, 16, 20, 22, 24, 28, 30, 32, 36, 38, and 40, and variants thereof. In some embodiments, a protein disclosed herein comprises at least two, three, four, five, six, seven, eight, nine, ten, eleven, or twelve sequences selected from SEQ ID NOs: 4, 6, 8, 12, 14, 16, 20, 22, 24, 28, 30, 32, 36, 38, and 40, and variants thereof.

In some embodiments, a TCR or protein disclosed herein comprises a CDR3 amino acid sequence selected from SEQ ID NOs: 8, 16, 24, 32, and 40, and variants thereof. In some embodiments, a TCR or protein disclosed herein comprises a CDR2 amino acid sequence selected from SEQ ID NOs: 6, 14, 22, 30, and 38, and variants thereof. In some embodiments, a TCR or protein disclosed herein comprises a CDR1 amino acid sequence selected from SEQ ID NOs: 4, 12, 20, 28, and 36, and variants thereof.

In some embodiments, a TCR or protein disclosed herein comprises SEQ ID NOs: 4, 6 and 8. In some embodiments, a TCR or protein disclosed herein comprises SEQ ID NOs: 12, 14 and 16. In some embodiments, a TCR or protein disclosed herein comprises SEQ ID NOs: 20, 22 and 24. In some embodiments, a TCR or protein disclosed herein comprises SEQ ID NOs: 28, 30 and 32. In some embodiments, a TCR or protein disclosed herein comprises SEQ ID NOs: 36, 38 and 40.

In some embodiments, a TCR or protein disclosed herein comprises the sequence of SEQ ID NO: 2, 18, 34, or a variant thereof, coding for the α-chain of the TCR, and/or the sequence of SEQ ID NO: 10, 26, or a variant thereof, coding for the β-chain of the TCR. In some embodiments, a variant is at least about 80%, 85%, 90%, 95%, or 99% identical to at least 60%, 70%, 80%, 90%, or 100% of SEQ ID NO: 2, 10, 18, 26, or 34, or has one, two, three, four, five, six, or seven amino acid differences from the recited sequences.

In some embodiments, the protein comprises SEQ ID NOs: 2 and 10. In some embodiments, the protein comprises SEQ ID NOs: 34 and 10. In some embodiments, the protein comprises SEQ ID NOs: 18 and 26.

The TCR or fragment thereof, polypeptide, or protein disclosed herein can consist essentially of the specified amino acid sequence or sequences described herein, such that other components e.g., other amino acids, do not materially change the biological activity of the functional variant.

The TCRs, fragments thereof, polypeptides, and proteins disclosed herein (including functional portions and functional variants) can be of any length, i.e., can comprise any number of amino acids, provided that the TCRs, polypeptides, or proteins (or functional portions or functional variants thereof) retain their biological activity, e.g., the ability to specifically bind to mutant IDH1 protein. For example, the polypeptide can be about 50 to about 5000 amino acids long, such as 50, 70, 75, 100, 125, 150, 175, 200, 300, 400, 500, 600, 700, 800, 900, 1000 or more amino acids in length. In this regard, the polypeptides disclosed herein also include oligopeptides.

The TCRs, fragments thereof, polypeptides, and proteins disclosed herein (including functional portions and functional variants) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, 3-phenyl serine 3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs, fragments thereof, polypeptides, and proteins disclosed herein (including functional portions and functional variants) can be, for example, glycosylated, amidated, carboxylated, phosphorylated, esterified, N-acylated, cyclized via, e.g., a disulfide bridge, or converted into an acid addition salt and/or optionally dimerized or polymerized, or conjugated.

When the TCRs, fragments thereof, polypeptides, and proteins disclosed herein (including functional portions and functional variants) are in the form of a salt, the polypeptides may be in the form of a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

A protein disclosed herein can be a recombinant antibody comprising at least one of the polypeptides described herein. As used herein, “recombinant antibody” refers to a recombinant (e.g., genetically engineered) protein comprising at least one of the polypeptides disclosed herein and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab′)2 fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with a polypeptide disclosed herein. The polypeptide of an antibody, or portion thereof, can be a polypeptide of any antibody or any antibody fragment, including any of the antibodies and antibody fragments described herein. In some embodiments, a recombinant antibody comprises a sequence selected from SEQ ID NOs: 4, 6, 8, 12, 14, 16, 20, 22, 24, 28, 30, 32, 36, 38, and 40, and variants thereof.

### Functional Variants

Included herein, in some embodiments, are functional variants of the inventive TCRs described herein. The term “functional variant” as used herein refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to a mutant R132H IDH1 polypeptide, or fragment thereof comprising the R132H mutation, for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, 50%, 75%, 80%, 90%, 95%, 96%, 97%, 98%, 99% or more identical in amino acid sequence to the parent TCR, polypeptide, or protein over the length of the variant. In some embodiments, a variant is at least about 80%, 85%, 90%, 95%, or 99% identical to at least 60%, 70%, 80%, 90%, or 100% of SEQ ID NO: 2, 10, 18, 26, or 34 over the length of the variant. In some embodiments, a variant has one, two, three, four, five, six, or seven amino acid changes compared to SEQ ID NO: 4, 6, 8, 12, 14, 16, 20, 22, 24, 28, 30, 32, 36, 38, or 40 over the length of the variant.

The functional variant can, for example, comprise the amino acid sequence of the parent TCR, fragment, polypeptide, or protein with at least one conservative amino acid substitution. Conservative amino acid substitutions are known in the art and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively, or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

### TCR Conjugates/Fusion Proteins

Included herein, in some embodiments, are conjugates, e.g., bioconjugates, comprising any of the inventive TCRs, polypeptides, or proteins disclosed herein (including any of the functional variants thereof). Conjugates, as well as methods of synthesizing conjugates in general, are known in the art (See, for instance, Hudecz, R, Methods Mol. Biol. 298: 209-223 (2005) and Kirin et al., Inorg Chem. 44(15): 5405-5415 (2005)).

In some embodiments, the T Cell Receptor (TCR) is associated with a detectable label, a therapeutic agent, a pharmacokinetic modifying moiety, or a combination of any of these.

In this regard, disclosed herein is a fusion protein comprising at least one of the polypeptides described herein fused with at least one other polypeptide. The other polypeptide can, for example, be expressed in frame (in tandem) with a polypeptide described herein. The other polypeptide can be any protein, or a portion thereof (e.g., a domain), including, but not limited to an immunoglobulin domain, CD3, CD4, CD8, an MHC molecule, a CD1 molecule, e.g., CD1a, CD1b, CD1c, CD1d, etc. The other polypeptide can encode a detectable label, such as an enzymatic label (for example, horseradish peroxidase, luciferase, alkaline phosphatase) or a predetermined polypeptide epitope recognized by a secondary reporter (for example, leucine zipper pair sequences, binding sites for secondary antibodies, metal binding domains, epitope tags, etc.).

Further, disclosed herein is a functional TCR α- and β-chain fusion protein. In some embodiments, a functional TCR α- and β-chain fusion protein is also fused to an epitope tag. In some embodiments, such a functional TCR α- and β-chain fusion protein comprises any of the exemplary polypeptide sequences disclosed herein.

### I. Nucleic Acids

A. Nucleic Acid Sequences Encoding IDH1

Disclosed herein, in some embodiments, is a nucleic acid comprising a nucleotide sequence that encodes any of the TCRs, polypeptides, and proteins herein. As such, disclosed herein is a nucleic acid molecule encoding an alpha and/or beta chain of a T-cell receptor (TCR) or a fragment thereof, wherein the TCR or fragment thereof binds to an R132H mutant of isocitrate dehydrogenase type 1 (IDH1) or a fragment of IDH1 comprising the R132H mutation, or a complementary nucleic acid molecule thereof. In some embodiments, the TCR, or fragment thereof, does not bind to IDH1 comprising an arginine at amino acid position 132. In some embodiments, the nucleotide sequence is codon-optimized or comprises a codon-optimized portion. The codon-optimized portion may comprise, consist, or consist essentially of an alpha or beta chain of a TCR disclosed herein, or a variable region thereof.

In some embodiments, the nucleic acids disclosed herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. Also disclosed herein is a nucleic acid coding for the T Cell Receptor (TCR) alpha and/or beta chain or for the T Cell Receptor (TCR), as disclosed herein.

B. Nucleic Acid Sequences and Variants

The nucleic acid can comprise variants thereof described herein. In some embodiments, the nucleotide sequence comprises, consists, or consists essentially of any of SEQ ID NOs: 1, 3, 5, 7, 9, 11, 13, 15, 17, 19, 21, 23, 25, 27, 29, 31, 33, 35, 37, and 39. In some embodiments, the nucleic acid comprises at least one of SEQ ID NOs: 1, 7, 9, 15, 17, 23, 25, 31, 33, and 39, and variants thereof. In some embodiments, a nucleic acid molecule disclosed herein comprises at least one sequence selected from SEQ ID NOs: 7, 15, 23, 31, and 39. In some embodiments, a nucleic acid molecule disclosed herein comprises a sequence selected from SEQ ID NOs: 3, 11, 19, 27, and 35, and variants thereof. In some embodiments, a nucleic acid molecule disclosed herein comprises a sequence selected from: SEQ ID NOs: 5, 13, 21, 29, and 37, and variants thereof.

In some embodiments, a nucleic acid molecule disclosed herein comprises SEQ ID NOs: 3, 5 and 7. In some embodiments, a nucleic acid molecule disclosed herein comprises SEQ ID NOs: 11, 13 and 15. In some embodiments, a nucleic acid molecule disclosed herein comprises SEQ ID NOs: 19, 21 and 23. In some embodiments, a nucleic acid molecule disclosed herein comprises SEQ ID NOs: 27, 29 and 31. In some embodiments, a nucleic acid molecule disclosed herein comprises SEQ ID NOs: 35, 37 and 39. In some embodiments, a nucleic acid molecule comprises the nucleic acid sequence of at least one of SEQ ID NOs: 1, 9, 17, 25, and 33.

In some embodiments, a nucleic acid molecule comprises SEQ ID NO: 1, or a variant thereof, and SEQ ID NO: 9, or a variant thereof. In some embodiments, a nucleic acid molecule comprises SEQ ID NO: 33, or a variant thereof, and SEQ ID NO: 9, or a variant thereof. In some embodiments, a nucleic acid molecule comprises SEQ ID NO: 17, or a variant thereof, and SEQ ID NO: 25, or a variant thereof. In some embodiments, a nucleic acid molecule comprises SEQ ID NOs: 1 and 9. In some embodiments, a nucleic acid molecule comprises SEQ ID NOs: 33 and 9. In some embodiments, a nucleic acid molecule comprises SEQ ID NOs: 17 and 25.

In some embodiments, a nucleic acid molecule disclosed herein comprises the nucleic acid sequence of at least one of SEQ ID NOs: 1, 9, 17, 25, and 33, and variants thereof. In some embodiments, a variant is at least 80%, 85%, 90%, 95%, or 99% identical to at least 60%, 70%, 80%, 90%, or 100% of SEQ ID NO: 1, 9, 17, or 25 over the length of the variant. Also disclosed herein is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein over the length of the variant. Likewise, variants may comprise from 1 to 21 nucleic acid differences from the recited SEQ ID NOs, over the length of the variant.

Also disclosed herein is a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand and are particularly suitable for detecting expression of any of the inventive TCRs (including functional portions and functional variants thereof). It is generally appreciated that conditions can be rendered more stringent by the addition of increasing amounts of formamide.

In some embodiments, the nucleotide sequence may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated with a tRNA that is more readily available within a cell, thus increasing translation efficiency. Optimization of the nucleotide sequence may also reduce secondary mRNA structures that would interfere with translation, thus increasing translation efficiency.

C. Natural and Non-Natural Nucleic Acids

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

The nucleic acids can be constructed based on chemical synthesis and/or enzymatic ligation reactions using procedures known in the art. In some embodiments, the nucleic acid comprises a non-natural nucleotide sequence. A nucleotide sequence may be considered to be “non-natural” if the nucleotide sequence is not found in nature. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyl uracil, uracil-5-oxyacetic acid methylester, 3-(3-amino-3-N-2-carboxypropyl) uracil, and 2,6-diaminopurine. Alternatively, one or more of the nucleic acids disclosed herein can be purchased from companies, such as Macromolecular Resources (Fort Collins, Colo.) and Synthegen (Houston, Tex.).

It is contemplated that all of the nucleic acid molecules disclosed herein can be isolated or substantially pure.

D. Vectors

The nucleic acids disclosed herein can be incorporated into a recombinant expression vector. In this regard, provided herein are recombinant expression vectors comprising any of the nucleic acids disclosed herein. In some embodiments, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors disclosed herein are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector disclosed herein can be any suitable recombinant expression vector and can be used to transform or transfect any suitable host cell. Suitable vectors include those designed for propagation and expansion or for expression or both, such as plasmids and viruses. In some embodiments, vectors include the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as XGTIQ, XGTI 1, λZa II (Stratagene), λEMBIA and λNMI 149, also can be used. Examples of plant expression vectors include pBIO1, pBI101.2, pBI101.3, pBI121 and pBIN19 (Clontech). Examples of animal expression vectors include pEUK-Cl, pMAM and pMAMneo (Clontech). In some embodiments, the recombinant expression vector is a viral vector, e.g., a retroviral or lentiviral vector. In an especially preferred embodiment, the recombinant expression vector is an MSGV1 vector.

The recombinant expression vectors disclosed herein can be prepared using standard recombinant DNA techniques. Constructs of expression vectors, which are circular or linear, can be prepared to contain a replication system functional in a prokaryotic or eukaryotic host cell. Replication systems can be derived, e.g., from ColEI, 2μ plasmid, SV40, bovine papillomavirus, and the like.

In some cases, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA or RNA based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter operably linked to the nucleotide sequence encoding the TCR, polypeptide, or protein (including functional variants thereof), or to the nucleotide sequence which is complementary to or which hybridizes to the nucleotide sequence encoding the TCR, polypeptide, or protein (including functional variants thereof). The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., β-actin promoter, SV40 early and late promoter, immunoglobulin promoter, human cytomegalovirus promoter, and retro viral LTRs. In some embodiments, the recombinant expression vector comprises an inducible or constitutive promoter.

The inventive recombinant expression vectors can be designed for either transient expression, for stable expression, or for both. Also, the recombinant expression vectors can be made for constitutive expression or for inducible expression. Further, the recombinant expression vectors can be made to include a suicide gene.

As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, and nitroreductase.

### II. Host Cells

A host cell is provided comprising any of the recombinant expression vectors or nucleic acid molecules described herein.

As used herein, the term “host cell” refers to any type of cell that can contain the disclosed recombinant expression vectors and nucleic acid molecules. In some embodiments, a host cell produces a polypeptide or TCR, as disclosed herein. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a. E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant TCR, polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). In some embodiments, said PBL is a T lymphocyte, or a cell capable of differentiating into a T lymphocyte.

For purposes herein, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th1 and Th2 cells, CD8⁺ T cells (e.g., cytotoxic T cells), peripheral blood mononuclear cells (PBMCs), peripheral blood leukocytes (PBLs), tumor infiltrating cells (TILs), memory T cells, naive T cells, and the like. The T cell may be a CD8⁺ T cell or a CD4⁺ T cell.

Also provided is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

### III. Methods of Production

The TCRs, polypeptides, and/or proteins disclosed herein (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the TCRs, polypeptides, and proteins disclosed herein (including functional portions and functional variants thereof) can be isolated and/or purified from a source, such as a plant, a bacterium, an insect, a mammal, e.g., a rat, a human, etc. Methods of isolation and purification are well-known in the art. Alternatively, the TCRs, polypeptides, and/or proteins described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.).

Also provided is a method to generate cells expressing the T Cell Receptor (TCR) as defined above comprising the following steps: a) activating a population of lymphocytes obtained from peripheral blood of a subject; b) isolating the T cells from said population; c) transducing, transfecting or transforming the isolated T cells with a nucleic acid as disclosed herein. In some embodiments, said nucleic acid is comprised in a vector.

In some methods of production, purification methods may be employed. The TCRs, polypeptides, proteins (including functional portions and variants thereof), nucleic acids, recombinant expression vectors, host cells (including populations thereof), and antibodies (including antigen binding portions thereof) disclosed herein can be isolated and/or purified. The term “isolated” as used herein means having been removed from its natural environment. The term “purified” or “isolated” does not require absolute purity or isolation; rather, it is intended as a relative term. Thus, for example, a purified (or isolated) protein preparation is one in which the protein is purer than the protein in its natural environment within a cell. Such proteins may be produced, for example, by standard purification techniques, or by recombinant expression. In some embodiments, a preparation of a protein is purified such that the protein represents at least 50%, for example at least 70%, of the total protein content of the preparation. For example, the purity can be at least about 50%, can be greater than about 60%, about 70% or about 80%, or can be about 100%.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

### IV. Pharmaceutical Compositions

The TCRs, polypeptides, proteins (including functional portions and variants thereof), nucleic acids, recombinant expression vectors, host cells (including populations thereof), and antibodies (including antigen binding portions thereof) disclosed herein can be formulated into a composition, such as a pharmaceutical composition. In this regard, provided herein is a pharmaceutical composition comprising any of the TCRs, polypeptides, proteins, functional portions, functional variants, nucleic acids, expression vectors, host cells (including populations thereof), and antibodies (including antigen binding portions thereof), and a pharmaceutically acceptable carrier. In some embodiments, the pharmaceutical compositions disclosed herein comprise more than one TCR material disclosed herein, e.g., a polypeptide and a nucleic acid (such as in a host cell comprising the nucleic acid and expressing the polypeptide), or two or more different TCRs. Alternatively, the pharmaceutical composition can comprise a TCR material disclosed herein in combination with another pharmaceutically active agents or drugs, such as a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

In some embodiments, a pharmaceutical composition comprises between 10¹ to 10¹⁰ host cells as disclosed herein, that produces a polypeptide or TCR, as disclosed herein.

In some embodiments, in a pharmaceutical composition disclosed herein, at least 25%, 30%, 35%, 40%, 45%, 50%, 55%, 60%, 65%, 70%, 75%, 80%, 75%, 80%, 85%, 90%, 95%, 99%, or 100% of the cells in the pharmaceutical composition are host cells as disclosed herein that produces a polypeptide or TCR, as disclosed herein. In some embodiments, in a pharmaceutical composition disclosed herein, at least 50% of the cells in the pharmaceutical composition are host cells as disclosed herein that produces a polypeptide or TCR, as disclosed herein. In some embodiments, in a pharmaceutical composition disclosed herein, at least 75% of the cells in the pharmaceutical composition are host cells as disclosed herein that produces a polypeptide or TCR, as disclosed herein. In some embodiments, in a pharmaceutical composition disclosed herein, at least 90% of the cells in the pharmaceutical composition are host cells as disclosed herein that produces a polypeptide or TCR, as disclosed herein. In some embodiments, in a pharmaceutical composition disclosed herein, at least 95% of the cells in the pharmaceutical composition are host cells as disclosed herein that produces a polypeptide or TCR, as disclosed herein. In some embodiments, in a pharmaceutical composition disclosed herein, at least 99% of the cells in the pharmaceutical composition are host cells as disclosed herein that produces a polypeptide or TCR, as disclosed herein.

One of ordinary skill in the art will readily appreciate that the TCR materials disclosed herein can be modified in any number of ways, such that the therapeutic or prophylactic efficacy of the inventive TCR materials is increased through the modification.

For instance, the inventive TCR materials can be conjugated either directly or indirectly through a linker to a targeting moiety. The practice of conjugating compounds, e.g., inventive TCR materials, to targeting moieties is known in the art. See, for instance, Wadhwa et al., J. Drug Targeting 3: 111-127 (1995) and U.S. Pat. No. 5,087,616. The term “targeting moiety” as used herein, refers to any molecule or agent that specifically recognizes and binds to a cell-surface receptor, such that the targeting moiety directs the delivery of the inventive TCR materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). The term “linker” as used herein, refers to any agent or molecule that bridges the inventive TCR materials to the targeting moiety. One of ordinary skill in the art recognizes that sites on the inventive TCR materials, which are not necessary for the function of the inventive TCR materials, are ideal sites for attaching a linker and/or a targeting moiety, provided that the linker and/or targeting moiety, once attached to the inventive TCR materials, do(es) not interfere with the function of the inventive TCR materials, i.e., the ability to bind to antigen, or to detect, treat, or prevent disease.

In some embodiments, disclosed herein is a pharmaceutical composition comprising a host cell, as described, and a pharmaceutically acceptable carrier and/or adjuvant. In some embodiments, the pharmaceutical composition comprises between 10¹ to 10¹⁰ host cells described herein.

Also disclosed herein is a pharmaceutical composition for use in the methods of treatment disclosed herein.

With respect to pharmaceutical compositions, the pharmaceutically acceptable carrier can be any of those conventionally used and is limited only by chemico-physical considerations, such as solubility and lack of reactivity with the active(s), and by the route of administration. The pharmaceutically acceptable carriers described herein, for example, vehicles, adjuvants, excipients, and diluents, are well-known to those skilled in the art and are readily available to the public. In some embodiments, the pharmaceutically acceptable carrier is one which is chemically inert to the active agent(s) and one which has no detrimental side effects or toxicity under the conditions of use.

The choice of carrier will be determined in part by the particular TCR material, as well as by the particular method used to administer the TCR material. Accordingly, there are a variety of suitable formulations of the pharmaceutical composition disclosed herein.

Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The concentration of TCR material in the pharmaceutical formulations can vary, e.g., from less than about 1%, usually at or at least about 10%, to as much as 20% to 50% or more by weight, and can be selected primarily by fluid volumes, and viscosities, in accordance with the particular mode of administration selected. Methods for preparing administrable (e.g., parenterally administrable) compositions are known or apparent to those skilled in the art and are described in more detail in, for example, Remington: The Science and Practice of Pharmacy, Lippincott Williams & Wilkins; 21st ed. (May 1, 2005).

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration as well as the existence, nature, and extent of any adverse side-effects that might accompany the administration of a particular active and the desired physiological effect. It will be appreciated by one of skill in the art that various diseases or disorders could require prolonged treatment involving multiple administrations, using the TCR materials in each or various rounds of administration.

The following formulations for oral, aerosol, parenteral (e.g., subcutaneous, intravenous, intraarterial, intramuscular, intradermal, interperitoneal, and intrathecal), and rectal administration are merely exemplary and are in no way limiting. More than one route can be used to administer the TCR materials, and in certain instances, a particular route can provide a more immediate and more effective response than another route.

Formulations suitable for parenteral administration include aqueous and nonaqueous isotonic sterile injection solutions, such as sterile water for injection, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, a pharmaceutical composition disclosed herein is an injectable formulation. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)). The inventive TCR material, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Additionally, the inventive TCR materials, or compositions comprising such inventive TCR materials, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

The amount or dose of the inventive TCR material administered should be sufficient to effect, e.g., a therapeutic or prophylactic response, in the subject or animal over a reasonable time frame. For example, the dose of the inventive TCR material should be sufficient to bind to antigen, or detect, treat or prevent disease.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the condition being treated.

Many assays for determining an administered dose are known in the art. In some embodiments, an assay, which comprises comparing the extent to which target cells are lysed or IFN-γ is secreted by T cells expressing the inventive TCR, polypeptide, or protein upon administration of a given dose of such T cells to a mammal among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

For purposes of the inventive methods, wherein host cells or populations of cells are administered to the host, the cells can be cells that are allogeneic or autologous to the host. The cells may be autologous to the host.

In addition to the aforedescribed pharmaceutical compositions, the inventive TCR materials can be formulated as inclusion complexes, such as cyclodextrin inclusion complexes, or liposomes. Liposomes can serve to target the inventive TCR materials to a particular tissue. Liposomes also can be used to increase the half-life of the inventive TCR materials. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The inventive composition can be used in conjunction with other therapeutic agents or therapies. Such systems can avoid repeated administrations of the inventive composition, thereby increasing convenience to the subject and the physician, and may be particularly suitable for certain composition embodiments disclosed herein.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

### V. Methods of Use

It is contemplated that the inventive pharmaceutical compositions, TCRs, polypeptides, proteins, nucleic acids, recombinant expression vectors, host cells, or populations of cells can be used in methods of treating or preventing a disease in a host. Without being bound to a particular theory, the inventive TCRs have biological activity, e.g., ability to recognize antigen, such that the TCR (or related inventive polypeptide or protein) when expressed by a cell is able to mediate an immune response against the cell expressing the antigen for which the TCR is specific. In this regard, disclosed herein is a method of treating or preventing a disease in a host, comprising administering to the host any of the pharmaceutical compositions in an amount effective to treat or prevent the disease in the host.

In some embodiments, disclosed herein, is a method of treating and/or preventing a cell proliferation-related disorder in a subject, comprising administering to the subject a pharmaceutical composition as disclosed herein. In some embodiments, the cell proliferation-related disorder can be any disorder wherein mutant IDH1 is expressed.

The term “cell proliferation related-disorder” or “cancer” as used herein refers to any malignant neoplasm resulting from the undesired growth and under certain conditions the invasion and/or metastasis of impaired cells in an organism. The cells giving rise to the cell proliferation related-disorder are genetically impaired and have usually lost their ability to control cell division, cell migration behavior, differentiation status and/or cell death machinery. Most cell proliferation related-disorders form a tumor, but some hematopoietic cancers, such as leukemia, do not. In some embodiments, the cell proliferation-related disorder is chosen from a glioma, a leukemia, prostate cancer, fibrosarcoma, paraganglioma, myelodysplasia or myelodysplastic syndrome. In some embodiments, said cell proliferation related-disorder is characterized by having a mutation in the genome of at least some cancer cells which results in the expression of a mutant IDH1 having the R132H mutation. Whether a cancer has a mutation in at least some of the cancer cells as specified before can be determined by the skilled artisan by PCR-based and/or sequencing based detection techniques.

In some embodiments, the cell proliferation-related disorder is a tumor of the CNS, such as a glioma. Gliomas have been reported to frequently comprise cells or even consist of cells comprising the aforementioned IDH1 mutation. In some embodiments, the said glioma is WHO II or WHO III astrocytoma, oligodendroglioma, oligoastrocytoma, glioblastoma, or gliosarcoma. In some embodiments, the glioma is a low-grade glioma. In some embodiments, the glioma comprises the R132H mutation of IDH1.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented.

In some embodiments, the pharmaceutical composition is administered intrathecally, epidurally, intracerebrally, or intracerebroventicularly.

The amounts or dose of the inventive TCR material administered should be sufficient to effect, e.g., a therapeutic or prophylactic response, in the subject or animal over a reasonable time frame. For example, the dose of the inventive TCR material should be sufficient to bind to antigen, or detect, treat or prevent disease in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration and the severity of the condition being treated. By way of example and not intending to limit the disclosure, the dose of the inventive TCR material can be about 0.001 to about 1000 mg/kg body weight of the subject being treated/day, from about 0.01 to about 10 mg/kg body weight/day, about 0.01 mg to about 1 mg/kg body weight/day.

Many assays for determining an administered dose are known in the art. In some embodiments, an assay, which comprises comparing the extent to which target cells are lysed or IFN-γ is secreted by T cells expressing the inventive TCR, polypeptide, or protein upon administration of a given dose of such T cells to a mammal among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

When the inventive TCR materials are administered with one or more additional therapeutic agents, one or more additional therapeutic agents can be coadministered to the mammal. By “coadministering” is meant administering one or more additional therapeutic agents and the inventive TCR materials sufficiently close in time such that the inventive TCR materials can enhance the effect of one or more additional therapeutic agents. In this regard, the inventive TCR materials can be administered first and the one or more additional therapeutic agents can be administered second, or vice versa. Alternatively, the inventive TCR materials and the one or more additional therapeutic agents can be administered simultaneously.

For purposes of the inventive methods, wherein host cells or populations of cells are administered to the host, the cells can be cells that are allogeneic or autologous to the host. The cells may be autologous to the host. In some embodiments, the cells autologous to the host T cells from the host, or T cells derived from cells from the host.

### VI. Kits

Disclosed herein, in some embodiments, is a kit comprising at least one pharmaceutical composition, TCR, polypeptide, protein, nucleic acid, recombinant expression vector, host cell, or population of cells as disclosed herein. In some embodiments, a kit comprises at least one vector coding for at least one polypeptide disclosed herein. In some embodiments, a kit comprises one or more vectors coding for a TCR, as disclosed herein.

As used herein, the term “kit” shall encompass an entity of physically separated components, which are intended for individual use, but in functional relation to each other. This means that the individual parts of the kit are provided for simultaneous or subsequent use or administration.

### VII. Exemplary Embodiments

The following are exemplary embodiments according to this disclosure:
1. An isolated and/or recombinantly engineered T Cell Receptor (TCR), or a fragment thereof, wherein said TCR or fragment thereof binds to a fragment of an isocitrate dehydrogenase type 1 (IDH1) protein comprising an R132H mutation, wherein said TCR comprises:
   a TRBV9 beta chain or a fragment thereof.
2. The TCR or fragment thereof of embodiment 1, wherein said TRBV9 beta chain comprises:
   a sequence encoded by some or all of a TRBV9*01 germline allele; and/or
   a glutamine amino acid residue (Q) at position 63.
3. The TCR or fragment thereof of embodiment 1 or 2 further comprising a TRAV26 alpha chain or a fragment thereof, optionally, wherein the TRAV26 alpha chain comprises a sequence encoded by some or all of a TRAV26-1*02 germline allele or a TRAV26-2*01 germline allele.
4. The TCR or fragment thereof of embodiment 1 or 2 further comprising a TRAV16 alpha chain or a fragment thereof, optionally, wherein the TRAV16 alpha chain comprises a sequence encoded by some or all of a TRBV16*01 germline allele.
5. The TCR or fragment thereof of embodiment 4, wherein the TRAV16 alpha chain comprises an asparagine amino acid residue (N) at position 81.
6. An isolated and/or recombinantly engineered T Cell Receptor (TCR), or a fragment thereof, wherein said TCR or fragment thereof binds to a fragment of an isocitrate dehydrogenase type 1 (IDH1) protein comprising an R132H mutation, wherein said TCR comprises:
   a TCR alpha chain or a fragment thereof comprising a CDR3 sequence of a TCR alpha chain polypeptide sequence of SEQ ID NO: 2, 18, 34, or a variant thereof differing by no more than one, two, three, or four amino acids within the TCR alpha chain CDR3 sequence; and/or
   a TCR beta chain or a fragment thereof comprising a CDR3 sequence of a TCR beta chain polypeptide sequence of SEQ ID NO: 10, 26, or a variant thereof differing by no more than one, two, three, or four amino acids within the TCR beta chain CDR3 sequence.
7. The TCR or fragment thereof of any one of embodiments 1 to 6, wherein said TCR or fragment thereof binds to a fragment of IDH1 comprising the R132H mutation when the fragment of IDH1 comprising the R132H mutation is bound to a major histocompatibility (MHC) class II molecule.
8. The TCR or fragment thereof of any one of embodiments 1, 2 and 5 to 7, wherein the TCR alpha chain or fragment thereof comprises the polypeptide sequence of SEQ ID NO: 8.
9. The TCR or fragment thereof of any one of embodiments 1 and 3 to 7, wherein the TCR alpha chain or fragment thereof comprises the polypeptide sequence of SEQ ID NO: 40.
10. The TCR or fragment thereof of any one of embodiments 1 to 9, wherein the TCR beta chain or fragment thereof comprises the polypeptide sequence of SEQ ID NO: 16.
11. The TCR or fragment thereof of embodiment 6 or 7, wherein the TCR alpha chain or fragment thereof comprises the polypeptide sequence of SEQ ID NO: 24.
12. The TCR or fragment thereof of embodiment 6, 7, or 11, wherein the TCR beta chain or fragment thereof comprises the polypeptide sequence of SEQ ID NO: 32.
13. The TCR or fragment thereof of any one of embodiments 1 to 12, further comprising at least one sequence chosen from:
   a CDR1 sequence of the TCR alpha chain polypeptide sequence of SEQ ID NOs: 2, 18, 34, or a variant thereof differing by no more than one, two, or three amino acids within the TCR alpha chain CDR1 sequence;
   a CDR2 sequence of the TCR alpha chain polypeptide sequence of SEQ ID NOs: 2, 18, 34, or a variant thereof differing by no more than one, two, three, or four amino acids within the TCR alpha chain CDR2 sequence;
   a CDR1 sequence of the TCR beta chain polypeptide sequence of SEQ ID NOs: 10, 26, or a variant thereof differing by no more than one, two, or three amino acids within the TCR beta chain CDR1 sequence; and
   a CDR2 sequence of the TCR beta chain polypeptide sequence of SEQ ID NOs: 10, 26, or a variant thereof differing by no more than one, two, three, or four amino acids within the TCR beta chain CDR2 sequence.
14. The TCR or fragment thereof of embodiment 13, wherein the TCR alpha chain or fragment thereof comprises the polypeptide sequence of SEQ ID NO: 4, 20, or 36.
15. The TCR or fragment thereof of embodiment 13 or 14, wherein the TCR alpha chain or fragment thereof comprises the polypeptide sequence of SEQ ID NO: 6, 22, or 38.
16. The TCR or fragment thereof of any one of embodiments 13 to 15, wherein the TCR beta chain or fragment thereof comprises the polypeptide sequence of SEQ ID NO: 12 or 28.
17. The TCR or fragment thereof of any one of embodiments 13 to 16, wherein the TCR beta chain or fragment thereof comprises the polypeptide sequence of SEQ ID NO: 14 or 30.
18. The TCR or fragment thereof of any one of embodiments 1, 2, and 5 to 17, wherein the TCR alpha chain or fragment thereof comprises the polypeptide sequences of each of SEQ ID NOs: 4, 6 and 8.
19. The TCR or fragment thereof of any one of embodiments 1 and 3 to 17, wherein the TCR alpha chain or fragment thereof comprises the polypeptide sequences of each of SEQ ID NOs: 36, 38, and 40.
20. The TCR or fragment thereof of any one of embodiments 1 to 19, wherein the TCR beta chain or fragment thereof comprises the polypeptide sequences of each of SEQ ID NOs: 12, 14 and 16.
21. The TCR or fragment thereof of any one of embodiments 5 to 17, wherein the TCR alpha chain or fragment thereof comprises the polypeptide sequences of each of SEQ ID NOs: 20, 22 and 24.
22. The TCR or fragment thereof of any one of embodiments 5 to 17 and 21, wherein the TCR beta chain or fragment thereof comprises the polypeptide sequences of each of SEQ ID NOs: 28, 30 and 32.
23. The TCR or fragment thereof of any one of embodiments 1 to 22, wherein the TCR alpha chain or fragment thereof comprises the polypeptide sequence of SEQ ID NO: 2, 18, 34, or a variant thereof, and/or wherein the TCR beta chain or fragment thereof comprises the polypeptide sequence of SEQ ID NO: 10, 26, or a variant thereof.
24. The TCR or fragment thereof of embodiment 23, wherein the variant is at least 80% identical (e.g., at least 85%, 90%, 95%, or 99% identical) to at least 60% (e.g., at least 70%, 80%, 90%, or 100%) of the polypeptide sequence of SEQ ID NO: 2, 10, 18, 26, or 34.
25. The TCR or fragment thereof of embodiment 24, wherein the TCR alpha chain comprises the polypeptide sequence of SEQ ID NO: 2.
26. The TCR or fragment thereof of embodiment 24, wherein the TCR alpha chain comprises the polypeptide sequence of SEQ ID NO: 34.
27. The TCR of fragment thereof of any one of embodiments 24 to 26, wherein the TCR beta chain comprises the polypeptide sequence of SEQ ID NO: 10.
28. The TCR or fragment thereof of embodiment 24, wherein the TCR alpha chain comprises the polypeptide sequence of SEQ ID NO: 18.
29. The TCR or fragment thereof of embodiment 24 or 28, wherein the TCR beta chain comprises the polypeptide sequence of SEQ ID NO: 26.
30. The TCR or fragment thereof of any one of embodiments 1 to 29, wherein the TCR, or fragment thereof, does not bind to IDH1 comprising an arginine at amino acid position 132, or to a fragment thereof comprising the arginine at amino acid position 132.
31. A nucleic acid molecule encoding the TCR or a fragment thereof of any one of embodiments 1 to 30.
32. An isolated and/or recombinantly engineered nucleic acid molecule encoding:
   a T-cell receptor (TCR) alpha chain, or a fragment thereof comprising a TCR alpha chain CDR3 polypeptide sequence, wherein the TCR alpha chain is encoded by a nucleic acid sequence of SEQ ID NO: 1, 17, 33, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides) within the sequence encoding the TCR alpha chain CDR3 polypeptide sequence; and/or
   a T-cell receptor (TCR) beta chain, or a fragment thereof comprising a TCR beta chain CDR3 polypeptide sequence, wherein the TCR beta chain is encoded by a nucleic acid sequence of SEQ ID NO: 9, 25, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides) within the sequence encoding the TCR beta chain CDR3 polypeptide sequence,
   wherein the TCR or fragment thereof comprising the TCR alpha chain and/or the TCR beta chain binds to a fragment of an isocitrate dehydrogenase type 1 (IDH1) protein comprising the R132H mutation.
33. The nucleic acid molecule of embodiment 32, wherein the TCR or fragment thereof binds to the fragment of IDH1 comprising the R132H mutation when the fragment of IDH1 comprising the R132H mutation is bound to a major histocompatibility (WIC) molecule.
34. The nucleic acid molecule of embodiment 32 or 33, wherein the TCR or fragment thereof does not bind to IDH1 or a fragment thereof comprising an arginine at amino acid position 132.
35. The nucleic acid molecule of any one of embodiments 32 to 34, wherein the TCR alpha chain is encoded by a nucleic acid sequence comprising the sequence of SEQ ID NO: 7, 23, 39, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
36. The nucleic acid molecule of any one of embodiments 32 to 35, wherein the TCR beta chain is encoded by a nucleic acid sequence comprising the sequence of SEQ ID NO: 15, 31, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
37. The nucleic acid molecule of any one of embodiments 32 to 36, wherein the TCR alpha chain is encoded by a nucleic acid sequence comprising the sequence of SEQ ID NO: 3, 19, 35, or a variant thereof differing by no more than 1, 2, 3, 4, 5, 6, 7, 8, 9, 10, 11, 12, 13, 14, or 15 nucleotides (or nucleosides).
38. The nucleic acid molecule of any one of embodiments 32 to 37, wherein the TCR alpha chain is encoded by a nucleic acid sequence comprising the sequence of SEQ ID NO: 5, 21, 37, or a variant thereof differing by no more than 1, 2, 3, 4, 5, 6, 7, 8, 9, 10, 11, 12, 13, 14, or 15 nucleotides (or nucleosides).
39. The nucleic acid molecule of any one of embodiments 32 to 38, wherein the TCR beta chain is encoded by a nucleic acid sequence comprising the sequence of SEQ ID NO: 11, 27, or a variant thereof differing by no more than 1, 2, 3, 4, 5, 6, 7, 8, 9, 10, 11, 12, 13, 14, or 15 nucleotides (or nucleosides).
40. The nucleic acid molecule of any one of embodiments 32 to 39, wherein the TCR beta chain is encoded by a nucleic acid sequence comprising the sequence of SEQ ID NO: 13, 29, or a variant thereof differing by no more than 1, 2, 3, 4, 5, 6, 7, 8, 9, 10, 11, 12, 13, 14, or 15 nucleotides (or nucleosides).
41. The nucleic acid molecule of any one of embodiments 32 to 40, wherein the TCR alpha chain is encoded by a nucleic acid sequence that comprises:
   the sequence of SEQ ID NO: 3, or a variant thereof differing by no more than 1, 2, 3, 4, 5, 6, 7, 8, 9, 10, 11, 12, 13, 14, or 15 nucleotides (or nucleosides);
   the sequence of SEQ ID NO: 5, or a variant thereof differing by no more than 1, 2, 3, 4, 5, 6, 7, 8, 9, 10, 11, or 12 nucleotides (or nucleosides); and
   the sequence of SEQ ID NO: 7, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
42. The nucleic acid molecule of any one of embodiments 32 to 40, wherein the TCR alpha chain is encoded by a nucleic acid sequence that comprises:
   the sequence of SEQ ID NO: 35, or a variant thereof differing by no more than 1, 2, 3, 4, 5, 6, 7, 8, 9, 10, 11, 12, 13, 14, or 15 nucleotides (or nucleosides);
   the sequence of SEQ ID NO: 37, or a variant thereof differing by no more than 1, 2, 3, 4, 5, 6, 7, 8, or 9 nucleotides (or nucleosides); and
   the sequence of SEQ ID NO: 39, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
43. The nucleic acid molecule of any one of embodiments 32 to 42, wherein the TCR beta chain is encoded by a nucleic acid sequence that comprises:
   the sequence of SEQ ID NO: 11, or a variant thereof differing by no more than 1, 2, 3, 4, 5, 6, 7, 8, 9, 10, 11, or 12 nucleotides (or nucleosides);
   the sequence of SEQ ID NO: 13, or a variant thereof differing by no more than 1, 2, 3, 4, 5, 6, 7, 8, 9, 10, 11, 12, 13, 14, or 15 nucleotides (or nucleosides); and
   the sequence of SEQ ID NO: 15, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
44. The nucleic acid molecule of any one of embodiments 32 to 40, wherein the TCR alpha chain is encoded by a nucleic acid sequence that comprises:
   the sequence of SEQ ID NO: 19, or a variant thereof differing by no more than 1, 2, 3, 4, 5, 6, 7, 8, 9, 10, 11, 12, 13, 14, or 15 nucleotides (or nucleosides);
   the sequence of SEQ ID NO: 21, or a variant thereof differing by no more than 1, 2, 3, 4, 5, 6, 7, 8, 9, 10, 11, 12, 13, 14, or 15 nucleotides (or nucleosides); and
   the sequence of SEQ ID NO: 23, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
45. The nucleic acid molecule of any one of embodiments 32 to 40 and 44, wherein the TCR beta chain is encoded by a nucleic acid sequence that comprises:
   the sequence of SEQ ID NO: 27, or a variant thereof differing by no more than 1, 2, 3, 4, 5, 6, 7, 8, 9, 10, 11, 12, 13, 14, or 15 nucleotides (or nucleosides);
   the sequence of SEQ ID NO: 29, or a variant thereof differing by no more than 1, 2, 3, 4, 5, 6, 7, 8, 9, 10, 11, 12, 13, 14, or 15 nucleotides (or nucleosides); and
   the sequence of SEQ ID NO: 31, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides).
46. The nucleic acid molecule of any one of embodiments 32 to 45, wherein the TCR alpha chain is encoded by a nucleic acid sequence comprising the sequence of SEQ ID NO: 1, 17, 33, or a variant thereof.
47. The nucleic acid molecule of any one of embodiments 32 to 46, wherein the TCR beta chain is encoded by a nucleic acid sequence comprising the sequence of SEQ ID NO: 9, 25, or a variant thereof.
48. The nucleic acid molecule of any one of embodiments 32 to 47, wherein the nucleic acid molecule comprises at least one variant, and wherein the variant is at least 80% identical (e.g., at least 85%, 90%, 95%, or 99% identical) to at least 60% (e.g., at least 70%, 80%, 90%, or 100%) of the sequence of SEQ ID NO: 1, 3, 5, 7, 9, 11, 13, 15, 17, 19, 21, 23, 25, 27, 29, 31, 33, 35, 37, or 39.
49. A nucleic acid molecule of any one of embodiments 32 to 48, wherein the nucleic acid molecule comprises at least one sequence chosen from SEQ ID NOs: 1, 3, 5, 7, 9, 11, 13, 15, 17, 19, 21, 23, 25, 27, 29, 31, 33, 35, 37, and 39.
50. The nucleic acid molecule of embodiment 49, wherein the TCR alpha chain is encoded by a nucleic acid sequence comprising the sequences of each of SEQ ID NOs: 3, 5 and 7.
51. The nucleic acid molecule of embodiment 49, wherein the TCR alpha chain is encoded by a nucleic acid sequence comprising the sequences of each of SEQ ID NOs: 35, 37 and 39.
52. The nucleic acid molecule of any one of embodiments 49 to 51, wherein the TCR beta chain is encoded by a nucleic acid sequence comprising the sequence of each of SEQ ID NOs: 11, 13 and 15.
53. The nucleic acid molecule of embodiment 49, wherein the TCR alpha chain is encoded by a nucleic acid sequence comprising the sequence of each of SEQ ID NOs: 19, 21 and 23.
54. The nucleic acid molecule of embodiment 49 or 53, wherein the TCR beta chain is encoded by a nucleic acid sequence comprising the sequence of each of SEQ ID NOs: 27, 29 and 31.
55. The nucleic acid molecule of any one of embodiments 32 to 34, comprising the sequence of SEQ ID NO: 1 and/or SEQ ID NO: 9.
56. The nucleic acid molecule of any one of embodiments 32 to 34, comprising the sequence of SEQ ID NO: 33 and/or SEQ ID NO: 9.
57. The nucleic acid molecule of any one of embodiments 32 to 34, comprising the sequence of SEQ ID NO: 17 and/or SEQ ID NO: 25.
58. The nucleic acid molecule of any of embodiments 31 to 57, wherein said nucleic acid molecule is isolated or substantially pure.
59. A recombinant expression vector comprising a nucleic acid of any one of embodiments 31 to 57.
60. The recombinant expression vector of embodiment 59, wherein said vector is a retroviral or lentiviral vector.
61. The recombinant expression vector of embodiment 59 or 60, comprising an inducible or constitutive promoter.
62. The recombinant expression vector of embodiment 61, wherein the promoter is a β-actin promoter, an SV40 early or late promoter, an immunoglobulin promoter, a human cytomegalovirus promoter, or a retroviral LTR promoter.
63. A host cell that produces a polypeptide or TCR of any one of embodiments 1 to 30.
64. A host cell transformed, transduced or transfected with the recombinant expression vector of any one of embodiments 59 to 62, or comprising a nucleic acid of any one of embodiments 31 to 57.
65. The host cell of embodiment 63 or 64, wherein said host cell is a peripheral blood lymphocyte.
66. The host cell of embodiment 65, wherein said peripheral blood lymphocyte is a T lymphocyte, or a cell capable of differentiating into a T lymphocyte.
67. A pharmaceutical composition comprising a host cell of any one of embodiments 63 to 66, and a pharmaceutically acceptable carrier and/or adjuvant.
68. The pharmaceutical composition of embodiment 67, comprising between 10³ to 10¹⁰ host cells of any one of embodiments 54 to 57.
69. The pharmaceutical composition of embodiment 67 or 68, wherein at least 50% of the cells in the pharmaceutical composition are host cells of any one of embodiments 63 to 66.
70. A method of generating cells expressing a T Cell Receptor (TCR) as defined in any one of embodiments 1 to 30 comprising the following steps:
   a. isolating T lymphocytes from a population of lymphocytes obtained from peripheral blood of a subject; and
   b. transducing, transfecting or transforming the isolated T lymphocytes with a nucleic acid molecule as embodimented in any of embodiments 31 to 58, or a vector as embodied in any of embodiments 59 to 62.
71. The method of embodiment 70, further comprising activating the isolated T lymphocytes obtained from the peripheral blood of the subject.
72. A method of treating a cell proliferation-related disorder in a subject, comprising administering to the subject a pharmaceutical composition as embodied in any of embodiments 67 to 69.
73. The method of embodiment 72, wherein the cells are derived from said subject.
74. The method of embodiment 73, wherein the cells derived from said subject are T lymphocytes.
75. The method of any one of embodiments 72 to 74, wherein the cell proliferation-related disorder is a tumor of the central nervous system (CNS).
76. The method of any of embodiments 72 to 74, wherein the cell proliferation-related disorder is chosen from a glioma, a leukemia, prostate cancer, fibrosarcoma, paraganglioma, myelodysplasia or myelodysplastic syndrome.
77. The method of embodiment 76, wherein the cell proliferation-related disorder is a glioma.
78. The method of embodiment 77, wherein the glioma is a low-grade glioma.
79. The method of embodiment 72 to 78, wherein the cell-proliferation disorder is associated with the IDH1 R132H mutation.
80. The method of any one of embodiments 72 to 79, wherein the pharmaceutical composition is administered to the subject subcutaneously, intradermally, intramuscularly, peritoneally, or intravenously.
81. The method of embodiments 80, wherein the pharmaceutical composition is administered intramuscularly.
82. The method of embodiment 80, wherein the pharmaceutical composition is administered intravenously.
83. The method of any of embodiments 72 to 79, wherein the pharmaceutical composition is administered intrathecally, epidurally, intracerebrally, or intracerebroventicularly.
84. A pharmaceutical composition for use in treating a cell-proliferation related disorder, comprising a host cell of any of embodiments 63 to 66, and a pharmaceutically acceptable carrier and/or adjuvant.
85. The pharmaceutical composition of embodiment 84, wherein the cell proliferation-related disorder is a tumor of the CNS.
86. The pharmaceutical composition of embodiment 84, wherein the cell proliferation-related disorder is chosen from a glioma, a leukemia, prostate cancer, fibrosarcoma, paraganglioma, myelodysplasia or myelodysplastic syndrome.
87. The pharmaceutical composition of embodiment 84, wherein the cell-proliferation related disorder is a glioma.
88. The pharmaceutical composition of embodiment 87, wherein the glioma is a low-grade glioma.
89. The pharmaceutical composition of any one of embodiments 84 to 88, wherein the cell-proliferation disorder is associated with the IDH1 R132H mutation.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. In some embodiments, “about” is inclusive of plus or minus 0.1%, 1%, 5% or 10% of a given value. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1: Exemplary TCR Sequences

TABLE 1
Table of Sequences

| SEQ | | |
|---|---|---|
| ID | Description of | |
| NO: | Sequence | Sequence |
| 1 | A1 alpha | ATGAGGCTGGTGGCAAGAGTAACTGTGTTTCTGACCTTTGGAACTAT |
| | nucleotide | AATTGATGCTAAGACCACCCAGCCCACCTCCATGGATTGCGCTGAAG |
| | sequence | GAAGAGCTGCAAACCTGCCTTGTAATCACTCTACCATCAGTGGAAAT |
| | (TRAV26) | GAGTATGTGTATTGGTATCGACAGATTCACTCCCAGGGGCCACAGTA |
| | | TATCATTCATGGTCTAAAAAACAATGAAACCAATGAAATGGCCTCTC |
| | | TGATCATCACAGAAGACAGAAAGTCCAGCACCTTGATCCTGCCCCAC |
| | | GCTACGCTGAGAGACACTGCTGTGTACTATTGCATCGTCAGAGTCTG |
| | | GAATGACATGCGCTTTGGAGCAGGGACCAGACTGACAGTAAAACCAA |
| | | ATATCCAGAACCCTGACCCTGCCGTGTACCAGCTGAGAGACTCTAAA |
| | | TCCAGTGACAAGTCTGTCTGCCTATTCACCGATTTTGATTCTCAAAC |
| | | AAATGTGTCACAAAGTAAGGATTCTGATGTGTATATCACAGACAAAA |
| | | CTGTGCTAGACATGAGGTCTATGGACTTCAAGAGCAACAGTGCTGTG |
| | | GCCTGGAGCAACAAATCTGACTTTGCATGTGCAAACGCCTTCAACAA |
| | | CAGCATTATTCCAGAAGACACCTTCTTCCCCAGCCCAGAAAGTTCCT |
| | | GTGATGTCAAGCTGGTCGAGAAAAGCTTTGAAACAGATACGAACCTA |
| | | AACTTTCAAAACCTGTCAGTGATTGGGTTCCGAATCCTCCTCCTGAA |
| | | AGTGGCCGGGTTTAATCTGCTCATGACGCTGCGGCTGTGGTCCAGC |
| 2 | A1 alpha | MRLVARVTVFLTFGTIIDAKTTQPTSMDCAEGRAANLPCNHSTISGN |
| | amino acid | EYVYWYRQIHSQGPQYIIHGLKNNETNEMASLIITEDRKSSTLILPH |
| | sequence | ATLRDTAVYYCIVRVWNDMRFGAGTRLTVKPNIQNPDPAVYQLRDSK |
| | (TRAV26) | SSDKSVCLFTDFDSQTNVSQSKDSDVYITDKTVLDMRSMDFKSNSAV |
| | | AWSNKSDFACANAFNNSIIPEDTFFPSPESSCDVKLVEKSFETDTNL |
| | | NFQNLSVIGFRILLLKVAGFNLLMTLRLWSS |
| 3 | A1 alpha | ACCATCAGTGGAAATGAGTAT |
| | CDR1 | |
| | nucleotide | |
| | sequence | |
| 4 | A1 alpha | TISGNEY |
| | CDR1 amino | |
| | acid sequence | |
| 5 | A1 alpha | GGTCTAAAAAACAAT |
| | CDR2 | |
| | nucleotide | |
| | sequence | |
| 6 | A1 alpha | GLKNN |
| | CDR2 amino | |
| | acid sequence | |
| 7 | A1 alpha | ATCGTCAGAGTCTGGAATGACATGCGC |
| | CDR3 | |
| | nucleotide | |
| | sequence | |
| 8 | A1 alpha | IVRVWNDMR |
| | CDR3 amino | |
| | acid sequence | |
| 9 | A1 beta | ATGGGCTTCAGGCTCCTCTGCTGTGTGGCCTTTTGTCTCCTGGGAGC |
| | nucleotide | AGGCCCAGTGGATTCTGGAGTCACACAAACCCCAAAGCACCTGATCA |
| | sequence | CAGCAACTGGACAGCGAGTGACGCTGAGATGCTCCCCTAGGTCTAGA |
| | (TRBV9) | GACCTCTCTGTGTACTGGTACCAACAGAGCCTGGACCAGGGCCTCCA |
| | | GTTCCTCATTCAGTATTATAATGGAGAAGAGAGAGCAAAAGGAAACA |
| | | TTCTTGAACGATTCTCCGCACAACAGTTCCCTGACTTGCACTCTGAA |
| | | CTAAACCTGAGCTCTCTGGAGCTGGGGGACTCAGCTTTGTATTTCTG |
| | | TGCCAGCAGCGTAGAACCCGGGACATCAGCCTACGAGCAGTACTTCG |
| | | GGCCGGGCACCAGGCTCACGGTCACAGAGGACCTGAAAAACGTGTTC |
| | | CCACCCGAGGTCGCTGTGTTTGAGCCATCAGAAGCAGAGATCTCCCA |
| | | CACCCAAAAGGCCACACTGGTGTGCCTGGCCACAGGCTTCTACCCCG |
| | | ACCACGTGGAGCTGAGCTGGTGGGTGAATGGGAAGGAGGTGCACAGT |
| | | GGGGTCAGCACAGACCCGCAGCCCCTCAAGGAGCAGCCCGCCCTCAA |
| | | TGACTCCAGATACTGCCTGAGCAGCCGCCTGAGGGTCTCGGCCACCT |
| | | TCTGGCAGAACCCCCGCAACCACTTCCGCTGTCAAGTCCAGTTCTAC |
| | | GGGCTCTCGGAGAATGACGAGTGGACCCAGGATAGGGCCAAACCTGT |
| | | CACCCAGATCGTCAGCGCCGAGGCCTGGGGTAGAGCAGACTGTGGCT |
| | | TCACCTCCGAGTCTTACCAGCAAGGGGTCCTGTCTGCCACCATCCTC |
| | | TATGAGATCTTGCTAGGGAAGGCCACCTTGTATGCCGTGCTGGTCAG |
| | | TGCCCTCGTGCTGATGGCCATGGTCAAGAGAAAGGATTCCAGAGGC |
| 10 | A1 beta amino | MGFRLLCCVAFCLLGAGPVDSGVTQTPKHLITATGQRVTLRCSPRSG |
| | acid sequence | DLSVYWYQQSLDQGLQFLIQYYNGEERAKGNILERFSAQQFPDLHSE |
| | (TRBV9) | LNLSSLELGDSALYFCASSVEPGTSAYEQYFGPGTRLTVTEDLKNVF |
| | | PPEVAVFEPSEAEISHTQKATLVCLATGFYPDHVELSWWVNGKEVHS |
| | | GVSTDPQPLKEQPALNDSRYCLSSRLRVSATFWQNPRNHFRCQVQFY |
| | | GLSENDEWTQDRAKPVTQIVSAEAWGRADCGFTSESYQQGVLSATIL |
| | | YEILLGKATLYAVLVSALVLMAMVKRKDSRG |
| 11 | A1 beta CDR1 | TCTAGAGACCTCTCT |
| | nucleotide | |
| | sequence | |
| 12 | A1 beta CDR1 | SRDLS |
| | amino acid | |
| | sequence | |
| 13 | A1 beta CDR2 | TATTATAATGGAGAAGAG |
| | nucleotide | |
| | sequence | |
| 14 | A1 beta CDR2 | YYNGEE |
| | amino acid | |
| | sequence | |
| 15 | A1 beta CDR3 | GCCAGCAGCGTAGAACCCGGGACATCAGCCTACGAGCAGTAC |
| | nucleotide | |
| | sequence | |
| 16 | A1 beta CDR3 | ASSVEPGTSAYEQY |
| | amino acid | |
| | sequence | |
| 17 | A3 alpha | ATGAAGACATTTGCTGGATTTTCGTTCCTGTTTTTGTGGCTGCAGCT |
| | nucleotide | GGACTGTATGAGTAGAGGAGAGGATGTGGAGCAGAGTCTTTTCCTGA |
| | sequence | GTGTCCGAGAGGGAGACAGCTCCGTTATAAACTGCACTTACACAGAC |
| | (TRAV5) | AGCTCCTCCACCTACTTATACTGGTATAAGCAAGAACCTGGAGCAGG |
| | | TCTCCAGTTGCTGACGTATATTTTTTCAAATATGGACATGAAACAAG |
| | | ACCAAAGACTCACTGTTCTATTGAATAAAAAGGATAAACATCTGTCT |
| | | CTGCGCATTGCAGACACCCAGACTGGGGACTCAGCTATCTACTTCTG |
| | | TGCAGAGAGGGGTAACAATGACATGCGCTTTGGAGCAGGGACCAGAC |
| | | TGACAGTAAAACCAAATATCCAGAACCCTGACCCTGCCGTGTACCAG |
| | | CTGAGAGACTCTAAATCCAGTGACAAGTCTGTCTGCCTATTCACCGA |
| | | TTTTGATTCTCAAACAAATGTGTCACAAAGTAAGGATTCTGATGTGT |
| | | ATATCACAGACAAAACTGTGCTAGACATGAGGTCTATGGACTTCAAG |
| | | AGCAACAGTGCTGTGGCCTGGAGCAACAAATCTGACTTTGCATGTGC |
| | | AAACGCCTTCAACAACAGCATTATTCCAGAAGACACCTTCTTCCCCA |
| | | GCCCAGAAAGTTCCTGTGATGTCAAGCTGGTCGAGAAAAGCTTTGAA |
| | | ACAGATACGAACCTAAACTTTCAAAACCTGTCAGTGATTGGGTTCCG |
| | | ATCCTCCTCCTGAAAGTGGCCGGGTTTAATCTGCTCATGACGCTGC |
| | | GGCTGTGGTCCAGC |
| 18 | A3 alpha | MKTFAGFSFLFLWLQLDCMSRGEDVEQSLFLSVREGDSSVINCTYTD |
| | amino acid | SSSTYLYWYKQEPGAGLQLLTYIFSNMDMKQDQRLTVLLNKKDKHLS |
| | sequence | LRIADTQTGDSAIYFCAERGNNDMRFGAGTRLTVKPNIQNPDPAVYQ |
| | (TRAV5) | LRDSKSSDKSVCLFTDFDSQTNVSQSKDSDVYITDKTVLDMRSMDFK |
| | | SNSAVAWSNKSDFACANAFNNSIIPEDTFFPSPESSCDVKLVEKSFE |
| | | TDTNLNFQNLSVIGFRILLLKVAGFNLLMTLRLWSS |
| 19 | A3 alpha | GACAGCTCCTCCACCTAC |
| | CDR1 | |
| | nucleotide | |
| | sequence | |
| 20 | A3 alpha | DSSSTY |
| | CDR1 amino | |
| | acid sequence | |
| 21 | A3 alpha | ATTTTTTCAAATATGGACATG |
| | CDR2 | |
| | nucleotide | |
| | sequence | |
| 22 | A3 alpha | IFSNMDM |
| | CDR2 amino | |
| | acid sequence | |
| 23 | A3 alpha | GCAGAGAGGGGTAACAATGACATGCGC |
| | CDR3 | |
| | nucleotide | |
| | sequence | |
| 24 | A3 alpha | AERGNNDMR |
| | CDR3 amino | |
| | acid sequence | |
| 25 | A3 beta | ATGCTGCTGCTTCTGCTGCTTCTGGGGCCAGGCTCCGGGCTTGGTGC |
| | nucleotide | TGTCGTCTCTCAACATCCGAGCTGGGTTATCTGTAAGAGTGGAACCT |
| | sequence | CTGTGAAGATCGAGTGCCGTTCCCTGGACTTTCAGGCCACAACTATG |
| | (TRBV20) | TTTTGGTATCGTCAGTTCCCGAAACAGAGTCTCATGCTGATGGCAAC |
| | | TTCCAATGAGGGCTCCAAGGCCACATACGAGCAAGGCGTCGAGAAGG |
| | | ACAAGTTTCTCATCAACCATGCAAGCCTGACCTTGTCCACTCTGACA |
| | | GTGACCAGTGCCCATCCTGAAGACAGCAGCTTCTACATCTGCAGTGC |
| | | TAGAGTGGGTTTCACCCCTACCTACGAGCAGTACTTCGGGCCGGGCA |
| | | CCAGGCTCACGGTCACAGAGGACCTGAAAAACGTGTTCCCACCCGAG |
| | | GTCGCTGTGTTTGAGCCATCAGAAGCAGAGATCTCCCACACCCAAAA |
| | | GGCCACACTGGTGTGCCTGGCCACAGGCTTCTACCCCGACCACGTGG |
| | | AGCTGAGCTGGTGGGTGAATGGGAAGGAGGTGCACAGTGGGGTCAGC |
| | | ACAGACCCGCAGCCCCTCAAGGAGCAGCCCGCCCTCAATGACTCCAG |
| | | ATACTGCCTGAGCAGCCGCCTGAGGGTCTCGGCCACCTTCTGGCAGA |
| | | ACCCCCGCAACCACTTCCGCTGTCAAGTCCAGTTCTACGGGCTCTCG |
| | | GAGAATGACGAGTGGACCCAGGATAGGGCCAAACCTGTCACCCAGAT |
| | | CGTCAGCGCCGAGGCCTGGGGTAGAGCAGACTGTGGCTTCACCTCCG |
| | | AGTCTTACCAGCAAGGGGTCCTGTCTGCCACCATCCTCTATGAGATC |
| | | TTGCTAGGGAAGGCCACCTTGTATGCCGTGCTGGTCAGTGCCCTCGT |
| | | GCTGATGGCCATGGTCAAGAGAAAGGATTCCAGAGGC |
| 26 | A3 beta amino | MLLLLLLLGPGSGLGAVVSQHPSWVICKSGTSVKIECRSLDFQATTM |
| | acid sequence | FWYRQFPKQSLMLMATSNEGSKATYEQGVEKDKFLINHASLTLSTLT |
| | (TRBV20) | VTSAHPEDSSFYICSARVGFTPTYEQYFGPGTRLTVTEDLKNVFPPE |
| | | VAVFEPSEAEISHTQKATLVCLATGFYPDHVELSWWVNGKEVHSGVS |
| | | TDPQPLKEQPALNDSRYCLSSRLRVSATFWQNPRNHFRCQVQFYGLS |
| | | ENDEWTQDRAKPVTQIVSAEAWGRADCGFTSESYQQGVLSATILYEI |
| | | LLGKATLYAVLVSALVLMAMVKRKDSRG |
| 27 | A3 beta CDR1 | GACTTTCAGGCCACAACT |
| | nucleotide | |
| | sequence | |
| 28 | A3 beta CDR1 | DFQATT |
| | amino acid | |
| | sequence | |
| 29 | A3 beta CDR2 | TCCAATGAGGGCTCCAAGGCC |
| | nucleotide | |
| | sequence | |
| 30 | A3 beta CDR2 | SNEGSKA |
| | amino acid | |
| | sequence | |
| 31 | A3 beta CDR3 | AGTGCTAGAGTGGGTTTCACCCCTACCTACGAGCAGTAC |
| | nucleotide | |
| | sequence | |
| 32 | A3 beta CDR3 | SARVGFTPTYEQY |
| | amino acid | |
| | sequence | |
| 33 | C1 alpha | ATGAAGCCCACCCTCATCTCAGTGCTTGTGATAATATTTATACTCAG |
| | nucleotide | AGGAACAAGAGCCCAGAGAGTGACTCAGCCCGAGAAGCTCCTCTCTG |
| | sequence | TCTTTAAAGGGGCCCCAGTGGAGCTGAAGTGCAACTATTCCTATTCT |
| | (TRAV16) | GGGAGTCCTGAACTCTTCTGGTATGTCCAGTACTCCAGACAACGCCT |
| | | CCAGTTACTCTTGAGACACATCTCTAGAGAGAGCATCAAAGGCTTCA |
| | | CTGCTAACCTTAACAAAGGCGAGACATCTTTCCACCTGAAGAAACCA |
| | | TTTGCTCAAGAGGAAGACTCAGCCATGTATTACTGTGCTCTAAGTAA |
| | | TGGCTTTCAGAAACTTGTATTTGGAACTGGCACCCGACTTCTGGTCA |
| | | GTCCAAATATCCAGAACCCTGACCCTGCCGTGTACCAGCTGAGAGAC |
| | | TCTAAATCCAGTGACAAGTCTGTCTGCCTATTCACCGATTTTGATTC |
| | | TCAAACAAATGTGTCACAAAGTAAGGATTCTGATGTGTATATCACAG |
| | | ACAAAACTGTGCTAGACATGAGGTCTATGGACTTCAAGAGCAACAGT |
| | | GCTGTGGCCTGGAGCAACAAATCTGACTTTGCATGTGCAAACGCCTT |
| | | CAACAACAGCATTATTCCAGAAGACACCTTCTTCCCCAGCCCAGAAA |
| | | GTTCCTGTGATGTCAAGCTGGTCGAGAAAAGCTTTGAAACAGATACG |
| | | AACCTAAACTTTCAAAACCTGTCAGTGATTGGGTTCCGAATCCTCCT |
| | | CCTGAAAGTGGCCGGGTTTAATCTGCTCATGACGCTGCGGCTGTGGT |
| | | CCAGC |
| 34 | C1 alpha | MKPTLISVLVIIFILRGTRAQRVTQPEKLLSVFKGAPVELKCNYSYS |
| | amino acid | GSPELFWYVQYSRQRLQLLLRHISRESIKGFTANLNKGETSFHLKKP |
| | sequence | FAQEEDSAMYYCALSNGFQKLVFGTGTRLLVSPNIQNPDPAVYQLRD |
| | (TRAV16) | SKSSDKSVCLFTDFDSQTNVSQSKDSDVYITDKTVLDMRSMDFKSNS |
| | | AVAWSNKSDFACANAFNNSIIPEDTFFPSPESSCDVKLVEKSFETDT |
| | | NLNFQNLSVIGFRILLLKVAGFNLLMTLRLWSS |
| 35 | C1 alpha | TATTCTGGGAGTCCTGAA |
| | CDR1 | |
| | nucleotide | |
| | sequence | |
| 36 | C1 alpha | YSGSPE |
| | CDR1 amino | |
| | acid sequence | |
| 37 | C1 alpha | CACATCTCTAGA |
| | CDR2 | |
| | nucleotide | |
| | sequence | |
| 38 | C1 alpha | HISR |
| | CDR2 amino | |
| | acid sequence | |
| 39 | C1 alpha | GCTCTAAGTAATGGCTTTCAGAAACTTGTA |
| | CDR3 | |
| | nucleotide | |
| | sequence | |
| 40 | C1 alpha | ALSNGFQKLV |
| | CDR3 amino | |
| | acid sequence | |
| 41 | IDH1 fragment | GWVKPIIIGHHAYGDQYRAT |
| | peptide | |
| | comprising | |
| | R132H | |
| | mutation, | |
| | wherein the | |
| | mutant | |
| | position is | |
| | underlined and | |
| | bold | |
| 42 | IDH1 wild | MSKKISGGSVVEMQGDEMTRIIWELIKEKLIFPYVELDLHSYDLGIE |
| | type amino | NRDATNDQVTKDAAEAIKKHNVGVKCATITPDEKRVEEFKLKQMWKS |
| | acid sequence | PNGTIRNILGGTVFREAIICKNIPRLVSGWVKPIIIGRHAYGDQYRA |
| | | TDFVVPGPGKVEITYTPSDGTQKVTYLVHNFEEGGGVAMGMYNQDKS |
| | | IEDFAHSSFQMALSKGWPLYLSTKNTILKKYDGRFKDIFQEIYDKQY |
| | | KSQFEAQKIWYEHRLIDDMVAQAMKSEGGFIWACKNYDGDVQSDSVA |
| | | QGYGSLGMMTSVLVCPDGKTVEAEAAHGTVTRHYRMYQKGQETSTNP |
| | | IASIFAWTRGLAHRAKLDNNKELAFFANALEEVSIETIEAGFMTKDL |
| | | ACIKGLPNVQRSDYLNTFEFMDKLGENLKIKLAQAKL |

### Example 2: T Cell Stimulation and Harvest

On day 1, peripheral blood mononuclear cells (PBMCs) were isolated from whole blood of a healthy human subject, HLADRB1*04:03⁺. The isolated PBMCs were seeded in the wells of a 24-well plate in RPMI containing 5% human serum, 10 U/mL interleukin 2 (IL-2) and 5 ng/mL interleukin 7 (IL-7). The medium was supplemented with 10 μg/mL IDH1 R132H mutant peptide antigen (GWVKPIIIGHHAYGDQYRAT; SEQ ID NO: 41). The IDH1 R132H mutant peptide was provided for uptake by dendritic cells, which could then stimulate the activation and proliferation of T cells having TCRs that specifically recognize the peptide.

On day 4, the cell culture medium was supplemented with IL-2, which was added to 10 U/mL, and IL-7, which was added to 5 ng/mL.

On day 10, 0.5 million frozen PBMCs (same donor) and additional IDH1 R132H mutant peptide antigen (10 μg/mL) were added to each well. The cell culture medium was also supplemented with IL-7, which was added to 5 ng/mL.

On days 11 and 14, the cell culture medium was again supplemented with additional IL-2, which was added to 10 U/mL, and IL-7, which was added to 5 ng/mL.

On day 17, 0.5 million PBMCs (same donor) and additional IDH1 R132H mutant peptide antigen (10 μg/mL) was added to each well. The cell culture medium was also supplemented with IL-7, which was added to 5 ng/mL.

On day 19, the cell culture medium was again supplemented with additional IL-2, which was added to 5 U/mL, and IL-7, which was added to 5 ng/mL.

On day 24, cells from two wells of the well plate were isolated based on evidence of cell growth, as assessed by the color of the cell culture medium changing from pink to yellow. The isolated cells were enriched for CD4⁺ T cells using the EasySep™ CD3 negative selection kit (Stemcell), the beads of which were supplemented with anti-CD8 magnetic microbeads (Miltenyi).

### Example 3: Antigen-Specific Expansion of Human T Cells in a Nanofluidic Device

On day 18 (relative to the protocol of Example 2), a frozen sample of ˜10 million PBMCs, obtained from the same human subject as in Example 2, was thawed. Human CD14⁺ monocytes were isolated from the thawed PBMCs using the EasySep™ Human Monocyte Enrichment Kit (Stemcell), then cultured for 7 days in DC culture medium (RPMI, 10% FBS, 2% Human AB serum, 100 ng/ml GM-CSF, 50 ng/ml IL-4) (R&D Systems) to promote differentiation of dendritic cells (DCs), substantially as described in Example 2 of International Patent Application PCT/US2017/22846, filed Mar. 16, 2017, the entire contents of which is incorporated herein by reference. On day 22, 250 ug/ml LPS (R&D Systems) was added to the culture medium to promote DC activation. At the same time as the addition of the LPS, the DCs were also pulsed with 10 μM IDH1 R132H mutant peptide antigen (GWVKPIIIGHHAYGDQYRAT; SEQ ID NO: 41).

On day 24, autologous donor CD4⁺ T lymphocytes (obtained in Example 2) were mixed with the IDH1 R132H mutant peptide antigen-pulsed DCs from the foregoing culture at a ratio of ˜10 T cells/1 DC and incubated for 5 hours in a 5% CO₂ incubator at 37° C. Following the incubation, the T cells/DCs mixture was resuspended and introduced into a nanofluidic chip configured with OptoSelect™ technology (Berkeley Lights, Inc.). The chip included a substrate configured with OptoElectroPositioning (OEP™) technology, which provides a light-actuated, phototransistor-activated OET force. The chip also included a plurality of microfluidic channels, each having a plurality of NanoPen™ chambers (or sequestration pens) fluidically connected thereto. The volume of each sequestration pen was around 1×10⁶ cubic microns (or 1 nL). The T cells/DCs resuspension was introduced into the chip by flowing the resuspension through a fluidic inlet and into the microfluidic channel. The flow was stopped and T cells/DCs were randomly loaded into the sequestration pens by tilting the chip and allowing gravity to pull the T cells/DCs into isolation regions located within the sequestration pens.

After loading the T cells/DCs into the sequestration pens, T cell culture medium (RPMI, 10% FBS, 2% Human AB serum, 50 U/ml IL2) (R&D Systems) was perfused through the microfluidic channel(s) of the chip for a period of 5 days. The sequestration pens and any T cells contained therein were imaged every 30 minutes for the entire 5-day culture period. Approximates 3% of the sequestration pens containing T cells exhibited T cell growth during the 5-day culture period, suggesting that the T cell growth was triggered in an antigen-specific manner.

T cells that expanded under the foregoing conditions were exported into individual wells of a well plate and their TCR genes were sequenced. TCR sequences from the cells of three such wells (A1, A3, and C1) are presented in Example 1, above. Sequencing of the cells in four of the wells resulted in the identification of more than one alpha subunit and more than one beta subunit, suggesting that the cells in those wells may have been polyclonal.

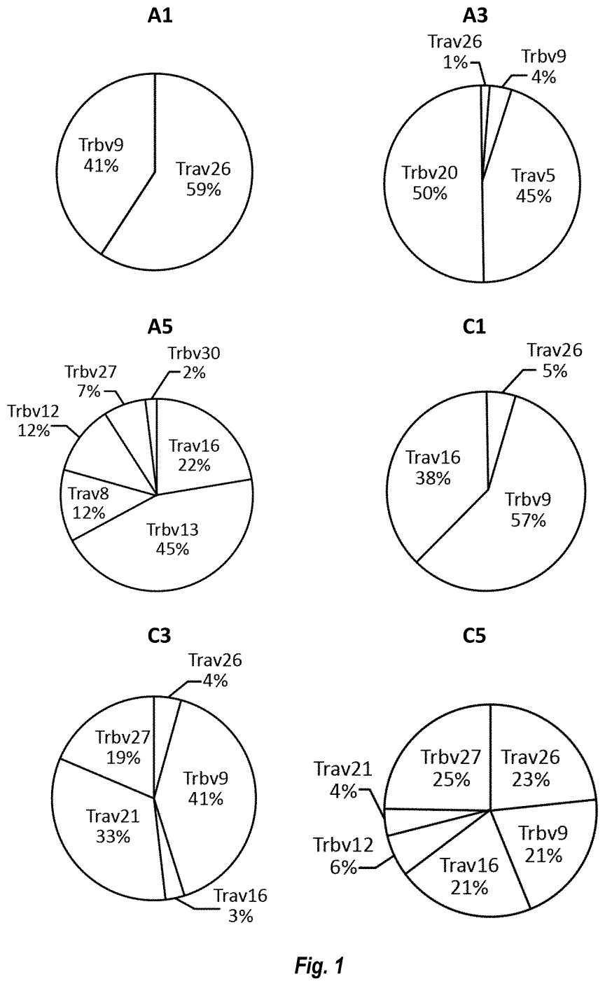

TRAV and TRBV allele frequencies from the exported T cells in six wells (A1, A3, A5, C1, C3, and C5) are shown in FIG. 1. Of note, cells from five of the six wells (A1, A3, C1, C3, and C5) included both a TRAV26 allele and a TRBV9 allele, and the TRBV9 alleles in wells A1 and C1 were identical. In addition, four of the six wells (A5, C1, C3, and C5) included a TRAV16 allele; and three of the six wells (A5, C3, and C5) included a TRBV27 allele. The results, along with the functional analysis of re-expressed TCRs described in Example 5—which shows that the TRBV9 allele of SEQ ID NO: 9 is functional when re-expressed with either the TRAV26 allele of SEQ ID NO: 1 or the TRAV16 allele of SEQ ID NO: 33—suggest that certain TCR alpha and beta chains, and possibly certain TCR alpha/beta chain combinations, have a greater propensity for binding to the IDH1 R132H mutant peptide.

In each of two subsequent experiments involving on-chip selection and expansion (as described above, in this Example 3) using T-cells from different donors, TCRs consisting of TRAV26-1 and TRBV9 were identified. The TRBV9 beta chains included distinct CDR3 sequences as compared to the CDR3 sequence (SEQ ID NO: 18) of the TRBV9 beta chain (SEQ ID NO: 10) identified in wells A1 and C1 of the experiment described above but arise from the same germline sequence (TRBV9*01), while the TRAV26 alpha chains arise from TRAV26-1*02 and TRAV26-2*01 germline sequences.

Taken together, it appears that the TRBV9 beta chain (optionally, comprising some or all of the amino acids encoded by a TRBV9*01 germline sequence) has a propensity for binding to the IDH1 R132H mutant peptide, while the TRAV26 alpha chain (optionally, comprising some or all of the amino acids encoded by a TRAV26-1*02 germline sequence or a TRAV26-2*01 germline sequence) and the TRAV16 alpha chains (optionally, comprising some or all of the amino acids encoded by a TRAV16*01 germline sequence, wherein the TRAV16 alpha chain may include an asparagine residue at position 81) are able to support/enhance the binding of the TRBV9 beta chain to the IDH1 R132H mutant peptide.

### Example 4: Transduction and Functional Activation of Isolated T-Cells from Murine Splenocytes

T cells isolated from murine splenocytes are transduced using retroviral vectors properly engineered and encoding a TCR specific for an R1321H IDH1 mutant, as described above in Example 1. Expression of the transduced TCR is assayed through cytofluorimetric analysis. The percentage of Vbl3 positive cells in the non-transduced splenocytes corresponds to the normal TCR variable repertoire, and the increase in this percentage in the transduced cells indicates that the cells have been successfully infected and express the R132H IDH1-specific TCR. The cytotoxic activity of the transduced cells against target cells loaded with the IDH1 R132H mutant peptide GWVKPIIIGHHAYGDQYRAT (SEQ ID NO: 41) is measured through a calcein release assay.

Transduced mouse splenocytes expressing an R132H IDH1-specific TCR, as described above, are analyzed for their ability to recognize target cells. In particular, the IFNγ production by transduced splenocytes cocultured with antigen presenting cells pre-pulsed with mutant R132H IDH1 mutant polypeptide is assayed. CD8⁺ splenocytes transduced with the retroviral vector encoding a R132H IDH1-specific TCR, as described herein, are expected to produce higher amounts of IFNγ when cocultured with R132H IDH1 antigen presenting cells, as compared to control transduced CD8⁺ T cells. Similarly, human CD8⁺ T cells transduced with a R132H IDH1-specific TCR, as described herein, are expected to produce higher amounts of IFNγ when cocultured with human antigen presenting cells that have been exposed to mutant R132H IDH1 polypeptide.

### Example 5: Functional Evaluation of Isolated TCRs

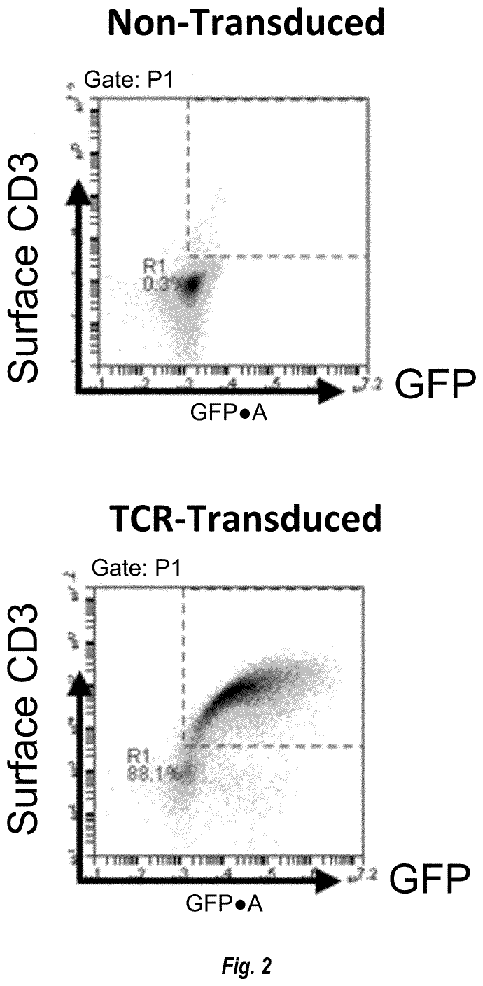

Jurkat 76 cells were transduced with TCR vectors engineered to express paired TCR α- and β-chains identified in Example 3 (above). Jurkat 76 cells are a human T cell lymphoma cell line that is deficient for endogenous TCR α and β chains, allowing a specific assessment of transgene TCR reactivity. As shown in FIG. 2, because expression of CD3 requires TCR α/β expression, non-transduced Jurkat 76 cells are negative for CD3. On the other hand, Jurkat 76 cells transduced with a TCR vector (GFP⁺ cells) express CD3 as a measure of accurate formation of TCR. The transduced Jurkat 76 cells shown in FIG. 1 express a TCR that includes the A1 α-chain (TRAV26-1, see Example 1) and the A1 β-chain (TRBV9, see Example 1), hereafter referred to as TCR clone 26-9. Two additional TCRs were successfully expressed in Jurkat 76 cells (data not shown), including: (i) TCR clone 5-20, containing the A3 α-chain (TRAV5, see Example 1) and the A3 β-chain (TRBV20-1, see Example 1); and (ii) TCR clone 16-9, containing the C1 α-chain (TRAV16) and the C1 β-chain (TRBV9).

CD4⁺ T-cells isolated from the peripheral blood of a HLADRB1* 01:01⁺ donor were transduced with lentiviral constructs encoding IDH1(R132H)-reactive TCRs identified in Example 3. Binding of the TCR-transduced CD4⁺ T-cells to HLA-DR1-IDH1(R132) tetramer was evaluated for all six TCR clones. TCR clones 26-9, 16-9, and 5-20, which produced the most reproducible positive staining against the tetramer (data not shown), were selected for further functional evaluations.

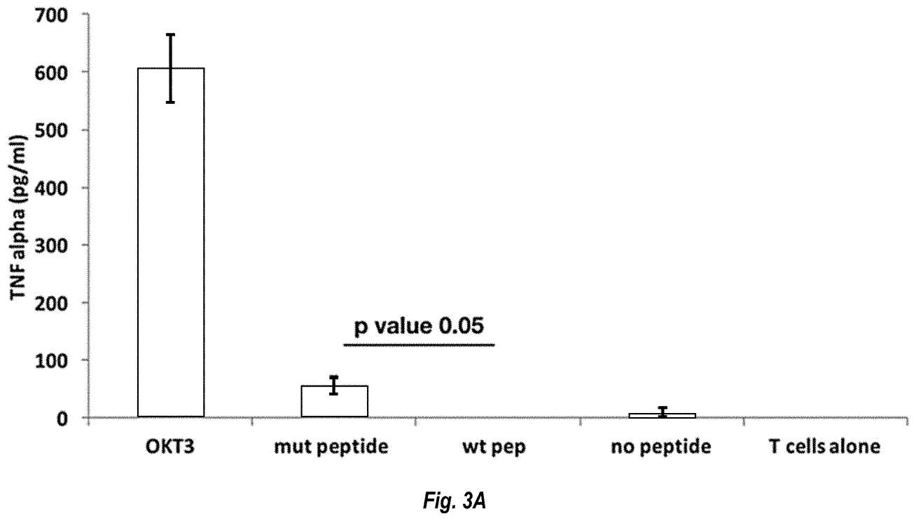

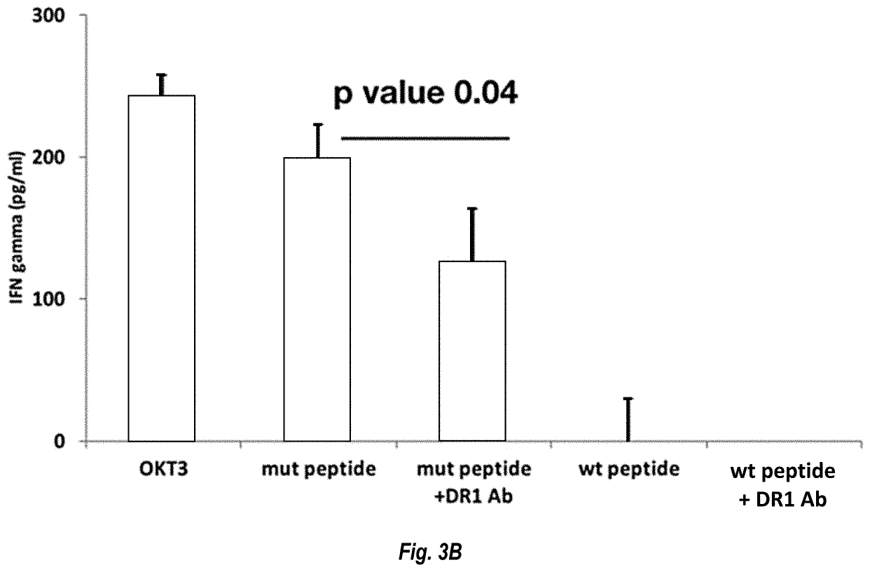

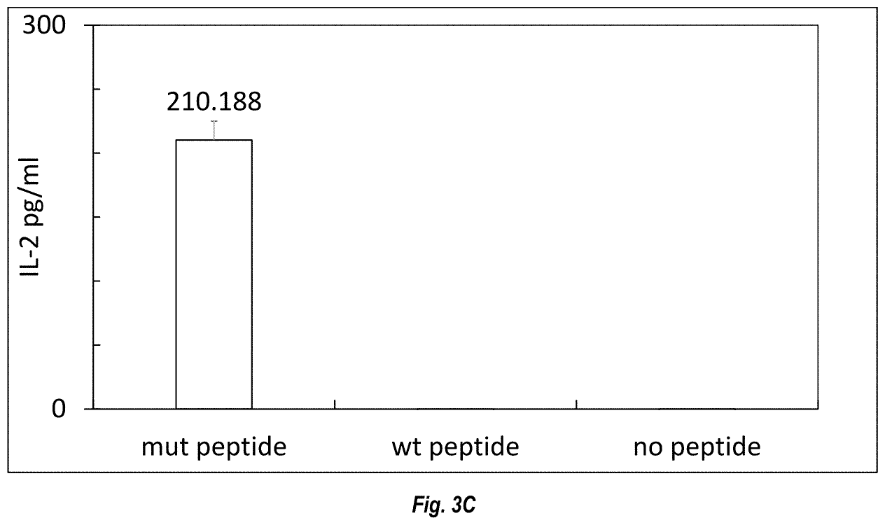

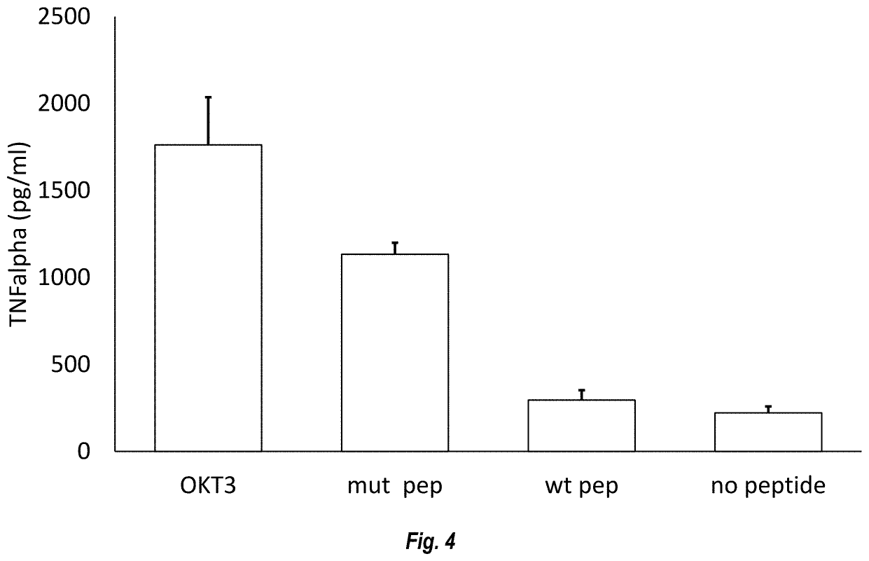

The TCR-transduced CD4⁺ T cells were co-cultured for 20 hours with autologous monocytes/B cells pulsed with 10 μg/ml IDH1 peptide (wild-type (wt) or containing the R132H mutation (mut)). Supernatants from the co-cultures were analyzed by ELISA for IFN-γ, TNF-α, and IL-2 production levels. IFN-γ levels were dramatically increased in supernatants for 26-9 and 5-20 clones when exposed to the IDH1(R132H;123-142) epitope peptide, as compared to exposure to control, wild-type peptide (FIG. 3B; shown for the 26-9 clone only). Furthermore, addition of an anti-HLA-DR-blocking antibody to the cultures significantly abrogated the production of IFN-γ (FIG. 3B). Exposure of the 26-9 and 16-9 TCR-transduced CD4⁺ T cells to the IDH1(R132H;123-142) epitope peptide also resulted in a significant increase in TNF-α supernatant levels as compared to exposure to the control, wild-type peptide (FIG. 3A, shown for the 26-9 clone only; FIG. 4), and a significant increase in IL-2 supernatant levels as compared to exposure to control, wild-type peptide (FIG. 3C, shown for the 26-9 clone only).

In the process of sequencing the alleles of the TCR clones of Example 3, amino acid substitutions within the TRBV9 and TRAV16 alleles were identified when compared with the corresponding annotated germline sequences for TRBV9 and TRAV16 (Tables 2 and 3, respectively). Mutation of the TRBV9 allele to switch Gln63 to Pro63 abrogated the function of the 26-9^{M} TCR upon re-expression (data not shown).

TABLE 2
Amino acid difference between TRBV9 (SEQ ID NO: 9; identified
in Example 3) and the annotated TRBV9*01 germline sequence.

| Annotated | 26-9 clone | 26-9 clone | 26-9^{M} clone | 26-9^{M} clone | Location |
|---|---|---|---|---|---|
| Amino acid | Amino acid | codon | Amino acid | codon | in V-domain |
| Pro63 | Gln63 | CAG | Pro63 | CCG | Between CDR1 and 2 |

TABLE 3
Amino acid difference between TRAV16 (SEQ ID NO: 33, identified
in Example 3) and the annotated TRAV16*01 germline sequence.

| Annotated | 16-9 clone | 16-9 clone | Location |
|---|---|---|---|
| Amino acid | Amino acid | codon | in V-domain |
| Asp81 | Asn81 | AAC | Between CDR2 and 3 |

Summary: NanoPen™ chambers within nanofluidic chips were used to selectively expand CD4⁺ T-cells that specifically recognize the IDH1(R132H) epitope peptide. Subsequently, full-length α and β chain cDNAs for 6 TCRs were successfully cloned, and the TCRs were successfully expressed in transfected host cells. CD4⁺ T-cells transduced with the 26-9 and 16-9 clones, in particular, produce effector cytokines (TNF-α and IFN-γ) in an epitope-specific manner.

The foregoing written specification is considered to be sufficient to enable one skilled in the art to practice the embodiments. The foregoing description and examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

1. An isolated and/or recombinantly engineered T Cell Receptor (TCR), or a fragment thereof, wherein said TCR or fragment thereof binds to a fragment of an isocitrate dehydrogenase type 1 (IDH1) protein comprising an R132H mutation, wherein said TCR comprises:
a TCR alpha chain or a fragment thereof comprising a CDR3 sequence of a TCR alpha chain polypeptide sequence of SEQ ID NO: 2, 18, 34, or a variant thereof differing by no more than one, two, three, or four amino acids within the TCR alpha chain CDR3 sequence; and/or
a TCR beta chain or a fragment thereof comprising a CDR3 sequence of a TCR beta chain polypeptide sequence of SEQ ID NO: 10, 26, or a variant thereof differing by no more than one, two, three, or four amino acids within the TCR beta chain CDR3 sequence.

2. The TCR or fragment thereof of claim 1, wherein said TCR or fragment thereof binds to a fragment of IDH1 comprising the R132H mutation when the fragment of IDH1 comprising the R132H mutation is bound to a major histocompatibility (MHC) class II molecule.

3. The TCR or fragment thereof of claim 1, wherein the TCR alpha chain or fragment thereof comprises the polypeptide sequence of SEQ ID NO: 8, 40, or 24, or the TCR beta chain or fragment thereof comprises the polypeptide sequence of SEQ ID NO: 16 or 32.

4.-7. (canceled)

8. The TCR or fragment thereof of claim 1, further comprising at least one sequence chosen from:
a CDR1 sequence of the TCR alpha chain polypeptide sequence of SEQ ID NOs: 2, 18, 34, or a variant thereof differing by no more than one, two, or three amino acids within the TCR alpha chain CDR1 sequence;
a CDR2 sequence of the TCR alpha chain polypeptide sequence of SEQ ID NOs: 2, 18, 34, or a variant thereof differing by no more than one, two, three, or four amino acids within the TCR alpha chain CDR2 sequence;
a CDR1 sequence of the TCR beta chain polypeptide sequence of SEQ ID NOs: 10, 26, or a variant thereof differing by no more than one, two, or three amino acids within the TCR beta chain CDR1 sequence; and
a CDR2 sequence of the TCR beta chain polypeptide sequence of SEQ ID NOs: 10, 26, or a variant thereof differing by no more than one, two, three, or four amino acids within the TCR beta chain CDR2 sequence.

9. The TCR or fragment thereof of claim 8, wherein the TCR alpha chain or fragment thereof comprises the polypeptide sequence of SEQ ID NO: 4, 20, 36, 6, 22, 38, or the TCR beta chain or fragment thereof comprises the polypeptide sequence of SEQ ID NO: 12, 28, 14, or 30.

10.-12. (canceled)

13. The TCR or fragment thereof of claim 1, wherein the TCR alpha chain or fragment thereof comprises the polypeptide sequences of each of SEQ ID NOs: 4, 6 and 8 and/or the TCR beta chain or fragment thereof comprises the polypeptide sequences of each of SEQ ID NOs: 12, 14 and 16.

14. The TCR or fragment thereof of claim 1, wherein the TCR alpha chain or fragment thereof comprises the polypeptide sequences of each of SEQ ID NOs: 36, 38 and 40 and/or the TCR beta chain or fragment thereof comprises the polypeptide sequences of each of SEQ ID NOs: 12, 14 and 16.

15. The TCR or fragment thereof of claim 1, wherein the TCR alpha chain or fragment thereof comprises the polypeptide sequences of each of SEQ ID NOs: 20, 22 and 24 and/or the TCR beta chain or fragment thereof comprises the polypeptide sequences of each of SEQ ID NOs: 28, 30 and 32.

16. The TCR or fragment thereof of claim 1, wherein the TCR alpha chain or fragment thereof comprises the polypeptide sequence of SEQ ID NO: 2, 18, 34, or a variant thereof, and/or wherein the TCR beta chain or fragment thereof comprises the polypeptide sequence of SEQ ID NO: 10, 26, or a variant thereof.

17. The TCR or fragment thereof of claim 16, wherein:
(i) the TCR alpha chain comprises the polypeptide sequence of SEQ ID NO: 2;
(ii) the TCR beta chain comprises the polypeptide sequence of SEQ ID NO: 10;
(iii) the TCR alpha chain comprises the polypeptide sequence of SEQ ID NO: 18;
(iv) the TCR beta chain comprises the polypeptide sequence of SEQ ID NO: 26;
(v) the TCR alpha chain comprises the polypeptide sequence of SEQ ID NO: 34
(vi) the TCR alpha chain comprises the polypeptide sequence of SEQ ID NO: 2 and the TCR beta chain comprises the polypeptide sequence of SEQ ID NO: 10;
(vii) the TCR alpha chain comprises the polypeptide sequence of SEQ ID NO: 34 and the TCR beta chain comprises the polypeptide sequence of SEQ ID NO: 10; or
(viii) the TCR alpha chain comprises the polypeptide sequence of SEQ ID NO: 18 and the TCR beta chain comprises the polypeptide sequence of SEQ ID NO: 26.

18. The TCR or fragment thereof of claim 1, wherein the TCR, or fragment thereof, does not bind to IDH1 comprising an arginine at amino acid position 132, or to a fragment thereof comprising the arginine at amino acid position 132.

19. A nucleic acid molecule encoding the TCR or a fragment thereof of claim 1.

20. An isolated and/or recombinantly engineered nucleic acid molecule encoding:
a T-cell receptor (TCR) alpha chain, or a fragment thereof comprising a TCR alpha chain CDR3 polypeptide sequence, wherein the TCR alpha chain is encoded by a nucleic acid sequence of SEQ ID NO: 1, 17, 33, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides) within the sequence encoding the TCR alpha chain CDR3 polypeptide sequence; and/or
a T-cell receptor (TCR) beta chain, or a fragment thereof comprising a TCR beta chain CDR3 polypeptide sequence, wherein the TCR beta chain is encoded by a nucleic acid sequence of SEQ ID NO: 9, 25, or a variant thereof differing by no more than 1, 2, 3, 4, 5, 6, 7, 8, 9, 10, 11, 12, 13, 14, 15, 16, 17, 18, 19, 20, or 21 nucleotides (or nucleosides) within the sequence encoding the TCR beta chain CDR3 polypeptide sequence,
wherein the TCR or fragment thereof comprising the TCR alpha chain and/or the TCR beta chain binds to a fragment of an isocitrate dehydrogenase type 1 (IDH1) protein comprising the R132H mutation.

21.-34. (canceled)

35. A host cell that produces a polypeptide or TCR of claim 1.

36.-40. (canceled)

41. A method of generating cells expressing a T Cell Receptor (TCR) as defined in claim 1 comprising the following steps:
a. isolating T lymphocytes from a population of lymphocytes obtained from peripheral blood of a subject; and
b. transducing, transfecting or transforming the isolated T lymphocytes with a nucleic acid molecule encoding the TCR, optionally wherein the nucleic acid molecule is an expression vector.

42. (canceled)

43. A method of treating a cell proliferation-related disorder in a subject, comprising administering to the subject a pharmaceutical composition comprising the host cell of claim 35, and a pharmaceutically acceptable carrier and/or adjuvant.

44. The method of claim 43, wherein the cells are derived from said subject, optionally, wherein the derived from said subject are T lymphocytes.

45.-48. (canceled)

49. An isolated and/or recombinantly engineered T Cell Receptor (TCR), or a fragment thereof, wherein said TCR or fragment thereof binds to a fragment of an isocitrate dehydrogenase type 1 (IDH1) protein comprising an R132H mutation, wherein said TCR comprises:
a TRBV9 TCR beta chain or a fragment thereof.

50. The TCR or fragment thereof of claim 49, wherein said TRBV9 beta chain comprises a glutamine amino acid residue (Q) at position 63.

51. The TCR or fragment thereof of claim 49 further comprising:
a TRAV26 TCR alpha chain or a fragment thereof; or
a TRAV16 TCR alpha chain or a fragment thereof, optionally wherein the TRAV16 TCR alpha chain comprises an asparagine amino acid residue (N) at position 81.


SEQUENCE LISTING (42 sequences)

SEQ ID NO: 1 - DNA, 798 bp, Artificial Sequence
  A1 alpha nucleotide sequence (TRAV26)
atgaggctggtggcaagagtaactgtgtttctgacctttggaactataattgatgctaagaccacccagcccacctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagtctggaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 2 - protein, 266 aa, Artificial Sequence
  A1 alpha amino acid sequence (TRAV26)
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVRVWNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - DNA, 21 bp, Artificial Sequence
  A1 alpha CDR1 nucleotide sequence
accatcagtggaaatgagtat

SEQ ID NO: 4 - protein, 7 aa, Artificial Sequence
  A1 alpha CDR1 amino acid sequence
TISGNEY

SEQ ID NO: 5 - DNA, 15 bp, Artificial Sequence
  A1 alpha CDR2 nucleotide sequence
ggtctaaaaaacaat

SEQ ID NO: 6 - protein, 5 aa, Artificial Sequence
  A1 alpha CDR2 amino acid sequence
GLKNN

SEQ ID NO: 7 - DNA, 27 bp, Artificial Sequence
  A1 alpha CDR3 nucleotide sequence
atcgtcagagtctggaatgacatgcgc

SEQ ID NO: 8 - protein, 9 aa, Artificial Sequence
  A1 alpha CDR3 amino acid sequence
IVRVWNDMR

SEQ ID NO: 9 - DNA, 939 bp, Artificial Sequence
  A1 beta nucleotide sequence (TRBV9)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctagagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagaacccgggacatcagcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 10 - protein, 313 aa, Artificial Sequence
  A1 beta amino acid sequence (TRBV9)
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVEPGTSAYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - DNA, 15 bp, Artificial Sequence
  A1 beta CDR1 nucleotide sequence
tctagagacctctct

SEQ ID NO: 12 - protein, 5 aa, Artificial Sequence
  A1 beta CDR1 amino acid sequence
SRDLS

SEQ ID NO: 13 - DNA, 18 bp, Artificial Sequence
  A1 beta CDR2 nucleotide sequence
tattataatggagaagag

SEQ ID NO: 14 - protein, 6 aa, Artificial Sequence
  A1 beta CDR2 amino acid sequence
YYNGEE

SEQ ID NO: 15 - DNA, 42 bp, Artificial Sequence
  A1 beta CDR3 nucleotide sequence
gccagcagcgtagaacccgggacatcagcctacgagcagtac

SEQ ID NO: 16 - protein, 14 aa, Artificial Sequence
  A1 beta CDR3 amino acid sequence
ASSVEPGTSAYEQY

SEQ ID NO: 17 - DNA, 813 bp, Artificial Sequence
  A3 alpha nucleotide sequence (TRAV5)
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagaggggtaacaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 18 - protein, 271 aa, Artificial Sequence
  A3 alpha amino acid sequence (TRAV5)
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERGNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - DNA, 18 bp, Artificial Sequence
  A3 alpha CDR1 nucleotide sequence
gacagctcctccacctac

SEQ ID NO: 20 - protein, 6 aa, Artificial Sequence
  A3 alpha CDR1 amino acid sequence
DSSSTY

SEQ ID NO: 21 - DNA, 21 bp, Artificial Sequence
  A3 alpha CDR2 nucleotide sequence
attttttcaaatatggacatg

SEQ ID NO: 22 - protein, 7 aa, Artificial Sequence
  A3 alpha CDR2 amino acid sequence
IFSNMDM

SEQ ID NO: 23 - DNA, 27 bp, Artificial Sequence
  A3 alpha CDR3 nucleotide sequence
gcagagaggggtaacaatgacatgcgc

SEQ ID NO: 24 - protein, 9 aa, Artificial Sequence
  A3 alpha CDR3 amino acid sequence
AERGNNDMR

SEQ ID NO: 25 - DNA, 930 bp, Artificial Sequence
  A3 beta nucleotide sequence (TRBV20)
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagtgggtttcacccctacctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 26 - protein, 310 aa, Artificial Sequence
  A3 beta amino acid sequence (TRBV20)
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARVGFTPTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 27 - DNA, 18 bp, Artificial Sequence
  A3 beta CDR1 nucleotide sequence
gactttcaggccacaact

SEQ ID NO: 28 - protein, 6 aa, Artificial Sequence
  A3 beta CDR1 amino acid sequence
DFQATT

SEQ ID NO: 29 - DNA, 21 bp, Artificial Sequence
  A3 beta CDR2 nucleotide sequence
tccaatgagggctccaaggcc

SEQ ID NO: 30 - protein, 7 aa, Artificial Sequence
  A3 beta CDR2 amino acid sequence
SNEGSKA

SEQ ID NO: 31 - DNA, 39 bp, Artificial Sequence
  A3 beta CDR3 nucleotide sequence
agtgctagagtgggtttcacccctacctacgagcagtac

SEQ ID NO: 32 - protein, 13 aa, Artificial Sequence
  A3 beta CDR3 amino acid sequence
SARVGFTPTYEQY

SEQ ID NO: 33 - DNA, 804 bp, Artificial Sequence
  C1 alpha nucleotide sequence (TRAV16)
atgaagcccaccctcatctcagtgcttgtgataatatttatactcagaggaacaagagcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctaaccttaacaaaggcgagacatctttccacctgaagaaaccatttgctcaagaggaagactcagccatgtattactgtgctctaagtaatggctttcagaaacttgtatttggaactggcacccgacttctggtcagtccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 34 - protein, 268 aa, Artificial Sequence
  C1 alpha amino acid sequence (TRAV16)
MKPTLISVLVIIFILRGTRAQRVTQPEKLLSVFKGAPVELKCNYSYSGSPELFWYVQYSRQRLQLLLRHISRESIKGFTANLNKGETSFHLKKPFAQEEDSAMYYCALSNGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - DNA, 18 bp, Artificial Sequence
  C1 alpha CDR1 nucleotide sequence
tattctgggagtcctgaa

SEQ ID NO: 36 - protein, 6 aa, Artificial Sequence
  C1 alpha CDR1 amino acid sequence
YSGSPE

SEQ ID NO: 37 - DNA, 12 bp, Artificial Sequence
  C1 alpha CDR2 nucleotide sequence
cacatctctaga

SEQ ID NO: 38 - protein, 4 aa, Artificial Sequence
  C1 alpha CDR2 amino acid sequence
HISR

SEQ ID NO: 39 - DNA, 30 bp, Artificial Sequence
  C1 alpha CDR3 nucleotide sequence
gctctaagtaatggctttcagaaacttgta

SEQ ID NO: 40 - protein, 10 aa, Artificial Sequence
  C1 alpha CDR3 amino acid sequence
ALSNGFQKLV

SEQ ID NO: 41 - protein, 20 aa, Homo sapiens
GWVKPIIIGHHAYGDQYRAT

SEQ ID NO: 42 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL